SEQUENCE LISTING (12 sequences)

SEQ ID NO: 1 - DNA, 1328 bp, Cucurbita ficifolia
  CDS (77)..(1060): SPDS
ccaacgggtcatacagaagcactccccactgtattgggatttgggattttagcgagtcgatagtagagggattattatgtctgcggaacatatcgttgggtcggcggccgatgcggcggcgaagaaacctgagattgagaatggggtatccgcctcacagcccgattctatttcctctgtaattcctggatggttttctgaaattagcccaatgtggcctggagaggcccattccttgaaggtggagaaggttttgtttcaagggaagtctgattaccagaacgttttggtatttcagtcatcaacttatgggaaggttctggttttggatggcgtgattcagcttacagagagagatgaatgtgcttaccaagagatgatcacccaccttccactttgctcaattccaaaccccaaaaaggttctcgttatcggtggaggagacggcggtgttttgcgagaggtggctcgccattcatctgttgagcagatagatatctgtgaaatcgacaagatggtagttgatgtttccaaagaatttttccctcgcgtagctgtcgggtttgaggatcctcgtgtcactcttcatattggtgatggcgtcgcatttctgaaggctgttcctgaaggcacttatgatgcagtgatagtggattcttctgatcctattggtcctgcacaagagctctttgagaagcctttttttgcttcagttgccaaagctcttcgaccaggaggcgttgtgtgtactcaagcagagagcatttggcttcacatgcatatcattgaagacattgtaacaaactgccgccaaatattcaaaggctctgtcaactatgcatggactacagttcctacatatccaagcggagtgattgggtttatgctctgctcaactgaggggcctcctcttgatttcaagcatccagtcaacccagtagaggtgaacggtatcgacaccgtgaagagtccgctcaagttttacaactcggagattcatacagcagctttctgtttgccttcttttgcgaagaagatcatcgattcaaaagcaaaatgaaaaggtttcccccacagcgttgaagaagcagaaattggcggtcttggagtgtgccaatgtaataagtggaggcttaaattagagtcgaaatggtcgctttatattgtgatcagcgtcataaagtttcttgagatgttatgagtagtagaaatagcttttgttttcctccccaaaattttccccgtcctttttcattgaaaagtgacatctggtgttctagcttctataaataaatatgctaaataaatatatttagccaaaaaaaaaa

SEQ ID NO: 2 - protein, 327 aa, Cucurbita ficifolia
MSAEHIVGSAADAAAKKPEIENGVSASQPDSISSVIPGWFSEISPMWPGEAHSLKVEKVLFQGKSDYQNVLVFQSSTYGKVLVLDGVIQLTERDECAYQEMITHLPLCSIPNPKKVLVIGGGDGGVLREVARHSSVEQIDICEIDKMVVDVSKEFFPRVAVGFEDPRVTLHIGDGVAFLKAVPEGTYDAVIVDSSDPIGPAQELFEKPFFASVAKALRPGGVVCTQAESIWLHMHIIEDIVTNCRQIFKGSVNYAWTTVPTYPSGVIGFMLCSTEGPPLDFKHPVNPVEVNGIDTVKSPLKFYNSEIHTAAFCLPSFAKKIIDSKAK

SEQ ID NO: 3 - DNA, 1560 bp, Oryza sativa
  CDS (118)..(1281): SPDS
gtgggccggagggcgagagtgaagcttgagccgtctcgccgcgggcgctgctgctgctgctgctgctcgcgtccgattccgacgagaggctgggaacttcaaagctgaaaaggactaatggagggtggaggcgcaaaagatggttctgcagcggcagcgcaaacaagggggagtggggatgatggttcccacaagccgctgccgccttgctgtctcaaggcgaaggctgcggcagcagagtctgaggcaaaatgccatgccacagtggtgtcagggtggttcacggaaccccgctcgcactctggtaaaacaagcaaagtgcagtacttcaacaatccaatgtggcctggagaagctcattctctgaaagtagagaatattctgtatcaagggaaatcaccataccaagaaatcttagtttttgagtcctcaacctatgggaatgttctggtgcttgatggtattgtgcagctgaccgaaaaggatgaatgtgcataccaggaaatggttactcaccttccactgtgctcgattccatcccccaaaagtgttttggttgtcggaggtggtgatggtggtgtgctacgagaaatagccagacatgcctcagtggagaacatcgatatatgtgaaatagaccagctagttattgatgtctgtaaagatttcttcccacaactatctgttggattcaaagatccccgtgttcaacttcatgttggtgacgctgtggatttcttgagaaatgcccctgaagggaaatatgatgctatcattgttgattcatctgacccaattgggccagctcaggagcttgtggagaagcccttttttcagacaattgctagagctctaaagcctggtggagttctatgcaatcaagctgagagtatgtggctgcacacacatctaattcaagatatgctttctatctgtcgtgaaacattcaagggagctgtgcattatgcctggacaagcgttcctacctatcctagtggtgtcattggttttttgctatgtgcgaaagaaggtccagcagtgaacttcctttctcctgtgaatccaattgagaaactggaaggagctatggaagctggaagggaaatcagattttacaattcagaggttcatcgggctgcttttgttctgccaacatttgtaaggagagaactggaatcacacaatacttgtgctgaaaaggataaatcagaaaccaagccagtcgcgaaaccaaagaagatgaaaataatgccgaatagtgccattccgactgcttcctagattcgggcggccatatgcgacaaccatgttcgacgtccttagctacagtatatgccaactataagaggagaatgtcgctgtaactcttctgtcttaagtatggtatgttatgcctgactgaataaaagggaggaacagatgagatgtgaaccaaaataatgttcttcctgatccgtgagcactgttgtagtgttgcctgttgtacaaatgtatattgtcacagcactgatgtgtttattatatggatgtggagtgaatcgcaaaaaaaaaaaaaaaaaa

SEQ ID NO: 4 - protein, 387 aa, Oryza sativa
MEGGGAKDGSAAAAQTRGSGDDGSHKPLPPCCLKAKAAAAESEAKCHATVVSGWFTEPRSHSGKTSKVQYFNNPMWPGEAHSLKVENILYQGKSPYQEILVFESSTYGNVLVLDGIVQLTEKDECAYQEMVTHLPLCSIPSPKSVLVVGGGDGGVLREIARHASVENIDICEIDQLVIDVCKDFFPQLSVGFKDPRVQLHVGDAVDFLRNAPEGKYDAIIVDSSDPIGPAQELVEKPFFQTIARALKPGGVLCNQAESMWLHTHLIQDMLSICRETFKGAVHYAWTSVPTYPSGVIGFLLCAKEGPAVNFLSPVNPIEKLEGAMEAGREIRFYNSEVHRAAFVLPTFVRRELESHNTCAEKDKSETKPVAKPKKMKIMPNSAIPTAS

SEQ ID NO: 5 - DNA, 1814 bp, Cucurbita ficifolia
  CDS (456)..(1547): SAMDC
tccgtctgcggcctcttgaattctcatcgtttctctctctttcgaattttgttttctttcgctctcagccctttcttgcaagttttattttaggcattctccgggtttccttctcctccgctaattcttttcatcgcgaatgatttaatggagtcaaaaggtggtaagaagtctagtagtagtagtagtagaagcagtaaatcccttttctacgaagctcccctcggatacagcattgaagacgttagaccacacggtggaatcaagaagttcagatctgctgcctactccaactgcgttcgtaaaccatcctgagttctgctgaattccgtttttcctgcgcaccgagatccttagttttctataatttttactgtgtcttttttctttagtactctactttcctcgttctctcgttcaatctctcaacattagtaacttccttttaagaaaagatgacgtttcctacctctgcaatcggatttgaaggctatgaaaagaggcttgaagtatcattctttgagcccggcatttttgctgacccaaggggcatgggccttcgtgctttgtccaaggcacaactagatgaaattctgacattagccgagtgcaccattgttgattctttgtccaatgactatcttgattcatatgtcctttcggagtcgagcctctttgtctacccatacaagttcatcatcaaaacttgcggcactactaagctgcttctgtctattccagctctgataaagttggctgattctctatcccttaatgtgaaatctgtgaggtacactcgtggaagctttatctttcctggtgcccagtcttttccccatcgcagcttctctgaggaagttgctgttcttgatggctacttggccaagcttggcctccatggctctgcttatgtgatgggaagtcctgatgagacaaggaaatggcacgtttactctgcctgtgccaaaatgggtagccgaagctacaatcccgtctatactctggagatgtgcatgactggcttagacaaggagaaggcgtctgtcttcttcaaaacagacacaagttctgctgctgcaatgactgaaaactccggtatcaggaagatccttccgaaatctgatatatgcgactttgagtttgacccatgtgggtattccatgaatgctattgaaggagatgcggagtctaccatccatgtcactccagaagaagggtttagctatgcaagctttgaagcagctggttatgaattggacgacctggacctgtgtaaggtgattgggagggtgctggcatgcttccagccatctgatttctctgttgccctccactcagatgtggtcggtgaggatctgaaagatttactgtgcctggacctgaaggggtacgagggtggagagaagagctgtgaaatgcttggggaaaatggatccgtcatctatcagagctttaagaatagaggagattatgcgtcatctccaaggtcaatcctcatgaaatgctgttggagagaggacgaggcggacgaggaagttgagaagtagtagtagttacttactttcaacttttgctgcgttttatcttttaatactatagtatcttcggggtcgttctgttctgtgctgttctgttctttcattatgtccttttgtgttgtttcctttgcgaataataattcccaggtggggatggtaggctgtcgtgtcctgtcctggagagtctatcgtctgatgttattatgatcatcaaactatataatgataatatcgtatttccttatttaaaaaaaaaaaaaaaaaaaaaaaaaaaaa

SEQ ID NO: 6 - protein, 363 aa, Cucurbita ficifolia
MTFPTSAIGFEGYEKRLEVSFFEPGIFADPRGMGLRALSKAQLDEILTLAECTIVDSLSNDYLDSYVLSESSLFVYPYKFIIKTCGTTKLLLSIPALIKLADSLSLNVKSVRYTRGSFIFPGAQSFPHRSFSEEVAVLDGYLAKLGLHGSAYVMGSPDETRKWHVYSACAKMGSRSYNPVYTLEMCMTGLDKEKASVFFKTDTSSAAAMTENSGIRKILPKSDICDFEFDPCGYSMNAIEGDAESTIHVTPEEGFSYASFEAAGYELDDLDLCKVIGRVLACFQPSDFSVALHSDVVGEDLKDLLCLDLKGYEGGEKSCEMLGENGSVIYQSFKNRGDYASSPRSILMKCCWREDEADEEVEK

SEQ ID NO: 7 - DNA, 3037 bp, Cucurbita ficifolia
  CDS (541)..(2661): ADC
gtttattaaacgcattctattgtctctccgagggctctccaattcttcccgttaggtttccgtttgttcctctttttctccgcctttttcccggaaaatctgtttgttgaagcaattgcatcctcttttgttttgtttttcttcttttgttgaatccctggtttgaatttttgtgtggattctttgatttccagatctgcatggtgaagagcggtttcggtgtttgatgtttgattggttttgaattcgtagcttgatttttgtgtttgttgttatcaaattcttcttctgagcggatcgcggtgggatattggaagtgtataggggagcgcggtggatttgacggtggaaatagctactttttttgcttcttttgagggggtagccggggcctcggcctcggcgggttttaaagcccccacttggacgaactctggatttaccattcctttctcttaacaatttctctaactaatcttttcgttttttaaattttccgtctccattttcttattcttttcttgatccgtcgtcggagagatgccggccctagcttattgcgtggaagctgctgcagctcctcctcctggctgcgcttttgctggggatagctctcttccgtcgccggtcttattttccggcggacctccggagactaccatcttcacctctcccgctgctgctcccatttctgaaaatccctcttggtctccttctctgtcttcctccctttacaagatagatggatggggtgccccttatttctctgtcaatggctctgggaatatggccgttcggccttacggtacagccaccttgccccatcaggagattgatctcttgaaaattgtgaagaaggcttcagatccgattagctctggtgggcttggcttgcagcttcctcttattgtgcgccttcctgatgtgcttaagaaccgtttggagtctctccaatcggcatttgattgtgctattcaatctcagggatatgggtctcattaccagggcgtttatccggtcaaatgcaaccaggacaggttcgttgttgaagacatcgtgaaattcggttctcctttccgtttcggtctcgaggctggatcgaaaccggagctcctcctggcaatgagctgtttgtgcaaagggaatagagatgcccttttggtgtgtaatggtttcaaggatgcggagtacatttctctggctcttattgctaggaagctcgctttgaacactgtgattgtgcttgaacaagaggaagagcttgatttggttatcgatttgagtaaaacgctcttcgttcgccctgtgatcggcatgcgtgcgaagctaagaaccaagcattctggtcattttgggtctacatcaggcgagaaagggaaatttggtcttacgaccacacaaattcttcgtgtggttaggaagcttaaacaggctgatatgcttgattgtcttcaattgctccattttcatattggttcccagatcccctccaccgtgttactcaccgatggcattagcgaggctgctcaaatctattgtgaattggttcgtctcggtgccaacatgctagttattgacattggaggtggtcttggtatcgactatgacgggtcgaagtcaggggattctgagttatctgttgcttatgaactcggagagtatgcctctacggttgttgatgcagtccgctgtgtatgcgaccgtagggccgttaagcacccgataatttgcagtgaaagtggccgagcaatcgtctctcatcactctgttctgatatttgaggctgtttctgctagttcttatgaggtcccatccatgagctcgattgaacgtcagtatcttgtcgatggactaaccgacgatgctcgtattgattatcagaaccttttgactgcagcttatatgggtgagtacaaggcgtgcttgctatatgcagatcaattgaagcaatgctgtgttgagaaattcaaggatgggtgtttgggaatggaagaactagctgcggtagatgggctttgtgcccttgtttcaaaggcaattggagagttggatgctgtaagaacttaccatgtgaacctctccattttcacctctatcccagatttctggggtattgaccagctgtttccaattgtccctattcatcgtctcgatcaaagaccgtcagtgaggggcattctatccgatctaacctgtgacagtgacggtaagatcgataggtttatcaatggcgagtcgagcttgccgttgcatgagctcaaaggcaacagcagtttatcaggtggaggtgggcgatactatcttgggatgtttctaggtggggcttatgaggaggctctcggtggtgttcacaacctgtttgggagcccgagcgtgattcgggtaatgcagagcgatggaccgcatagctttgcggtgactcgcactgtgcctgggccatcatgtgcggatatcctccgagtgatgcagtacgagcccgagctcatgtttgagaccctcaagcatcgagctgaggagtttgggcaggaggaggaggatgatgttggaggcattgccaatagcttggccatgtccttccgcaacatgccttatttggctagcgcatcttcctgcgccaatggtgctggcgatgccgagcagtggacttactgctatgcttgatgaataatgtttgaaggtttagtcgttagccacatccctaaataagctattggtctgttttcgttgtcgtggtcgtcgtcgtcgtaggtccgtcaacctttttttttttcttctttggcttgttgcaaagggttatgagagcacagcaacagcagccaagctcctcttcctttggctttatttttgtttagataggagaggggattagtagaacaccgaatccacccttttgttaattcgggatcttgatctctcttggttatatcatggtgtacaacttttaagaagccgtcaatggctgtttttcttttagatctcaactttggatggctcaaccccacttcgaattataaaaaaaaaaaaaaaaaaaaaaaaa

SEQ ID NO: 8 - protein, 706 aa, Cucurbita ficifolia
MPALAYCVEAAAAPPPGCAFAGDSSLPSPVLFSGGPPETTIFTSPAAAPISENPSWSPSLSSSLYKIDGWGAPYFSVNGSGNMAVRPYGTATLPHQEIDLLKIVKKASDPISSGGLGLQLPLIVRLPDVLKNRLESLQSAFDCAIQSQGYGSHYQGVYPVKCNQDRFVVEDIVKFGSPFRFGLEAGSKPELLLAMSCLCKGNRDALLVCNGFKDAEYISLALIARKLALNTVIVLEQEEELDLVIDLSKTLFVRPVIGMRAKLRTKHSGHFGSTSGEKGKFGLTTTQILRVVRKLKQADMLDCLQLLHFHIGSQIPSTVLLTDGISEAAQIYCELVRLGANMLVIDIGGGLGIDYDGSKSGDSELSVAYELGEYASTVVDAVRCVCDRRAVKHPIICSESGRAIVSHHSVLIFEAVSASSYEVPSMSSIERQYLVDGLTDDARIDYQNLLTAAYMGEYKACLLYADQLKQCCVEKFKDGCLGMEELAAVDGLCALVSKAIGELDAVRTYHVNLSIFTSIPDFWGIDQLFPIVPIHRLDQRPSVRGILSDLTCDSDGKIDRFINGESSLPLHELKGNSSLSGGGGRYYLGMFLGGAYEEALGGVHNLFGSPSVIRVMQSDGPHSFAVTRTVPGPSCADILRVMQYEPELMFETLKHRAEEFGQEEEDDVGGIANSLAMSFRNMPYLASASSCANGAGDAEQWTYCYA

SEQ ID NO: 9 - DNA, 1020 bp, Arabidopsis thaliana
  CDS (1)..(1020): SPMS
atgggtgaagccgtagaggtcatgttcggaaatgggttcccggagattcacaaagccacatcacccactcaaaccctccactctaaccagcaagactgccattggtatgaagaaaccatcgatgatgatctcaagtggtcttttgccctcaacagtgttctccatcaaggaactagtgagtaccaagatattgctctgttggacaccaaacgttttggaaaggtgcttgtgattgatgggaaaatgcaaagtgctgagagagatgagtttatctaccatgaatgtttgatccatcccgctctccttttccatcccaaccccaagactgtgtttataatgggaggaggtgaaggctctgctgcaagagaaatactaaaacacacgacgatcgagaaagttgttatgtgtgatattgatcaggaagttgttgatttttgcagaagatttctgaccgttaacagcgatgctttctgtaacaaaaagcttgaacttgtgatcaaagatgcaaaggctgaattagagaaaagggaagagaagtttgatatcatagtgggagatttagctgatccagtggaaggtggaccttgttatcagctctacaccaaatctttctaccaaaacattctcaaacccaagcttagccctaatggcatttttgtcacccaggctggaccagcaggaatattcactcataaggaagtcttcacatcaatctacaacaccatgaagcaagtcttcaagtacgtgaaggcttacacagcacatgtgccatcatttgcggacacatggggatgggtgatggcatcggaccacgagtttgacgttgaagttgatgaaatggatcgaagaatcgaagagagagttaacggagaattgatgtatctaaacgctccttctttcgtctctgctgctactctcaacaaaaccatctctctcgcgctagagaaggagactgaagtttatagtgaagagaatgcgagattcattcatggtcatggtgtggcgtaccggcatatttaa

SEQ ID NO: 10 - protein, 339 aa, Arabidopsis thaliana
MGEAVEVMFGNGFPEIHKATSPTQTLHSNQQDCHWYEETIDDDLKWSFALNSVLHQGTSEYQDIALLDTKRFGKVLVIDGKMQSAERDEFIYHECLIHPALLFHPNPKTVFIMGGGEGSAAREILKHTTIEKVVMCDIDQEVVDFCRRFLTVNSDAFCNKKLELVIKDAKAELEKREEKFDIIVGDLADPVEGGPCYQLYTKSFYQNILKPKLSPNGIFVTQAGPAGIFTHKEVFTSIYNTMKQVFKYVKAYTAHVPSFADTWGWVMASDHEFDVEVDEMDRRIEERVNGELMYLNAPSFVSAATLNKTISLALEKETEVYSEENARFIHGHGVAYRHI

SEQ ID NO: 11 - DNA, 20 bp, Artificial Sequence
  Description of Artificial Sequenceprimer
gttttggatggagtgattca

SEQ ID NO: 12 - DNA, 18 bp, Artificial Sequence
  Description of Artificial Sequenceprimer
gtgaatctcagcgttgta

(19) United States
(12) Patent Application Publication
(10) Pub. No.: US 2006/0225154 A1
(43) Pub. Date: Oct. 5, 2006

(54) Method for increasing expression of stress defense genes

(76) Inventors: Yoshihisa Kasukabe, Otsu-shi (JP); Atsushi Sogabe, Otsu-shi (JP); Izumi Ihara, Otsu-shi (JP); Tachibana Shoji, Tsu-shi (JP)

Correspondence Address: KENYON & KENYON LLP, 1500 K STREET N.W., SUITE 700, WASHINGTON, DC, 20005, US

(21) Appl. No.: 11/372,036

(22) Filed: Mar. 10, 2006

Related U.S. Application Data
(60) Provisional application No. 60/666,177, filed on Mar. 30, 2005.
(60) Provisional application No. 60/685,490, filed on May 31, 2005.

(30) Foreign Application Priority Data
Jun. 21, 2005 (JP) 2005-180644

(51) Int. Cl. A01H 1/00; C12N 15/82

(52) U.S. Cl. 800/289; 435/468

(57) ABSTRACT

The present invention provide methods of imparting stress tolerance, characterized in that an expression amount of at least one stress defense gene is increased compared with a non-transformant by transforming the plant with an exogenous spermidine synthase (SPDS) gene, an exogenous S-adenosylmethionine decarboxylase (SAMDC) gene, an exogenous arginine decarboxylase (ADC) gene, an ornithine decarboxylase (ODC) gene and/or a spermine synthase (SPMS) gene under the control of a promoter capable of functioning in the plant.

### TECHNICAL FIELD

The present invention relates to a method of increasing expression amounts of stress defense genes involved in stress tolerance or stress resistance in plants, and a method of imparting various stress defense effects by increasing the expression amounts of the stress defense genes involved in the stress tolerance or the stress resistance.

### BACKGROUND ART

Plants adapt to various types of environmental stress such as the temperature and salt of their habitats. However, in terms of temperature stress, for example, plants are susceptible to hot or cold temperatures when exposed to environments over or under the maximum or minimum optimum growth temperature, leading to impairment upon the gradual or sudden loss of the physiological functions of cells. Efforts have been made to expand the temperature adaptability of plants by breeding means such as selection or cross breeding in order to make use of wild plants adapted to various temperature environments for food crops, horticultural plants, and the like. The planting period in which vegetables, flowers and ornamental plants, fruit trees, and the like can be cultivated has been expanded by such breeding means as well as by protected horticulture. However, Japan in particular extends a considerable length to the north and south, with extreme variation in climate and considerable change in seasons from area to area, resulting in a greater risk of crop exposure to temperature environments that are not conducive to growth, depending on the area and season. Rice, for example, which originates in tropical regions, can now be cultivated in cooler areas such as the Tohoku district and Hokkaido as a result of improvements in varieties since the Meiji period, and are now cultivated as staples of these regions, but unseasonably low temperatures in early summer in these areas recently have resulted in cold-weather damage, leading to the problem of severe shortages even now. Recently, abnormal atmospheric phenomena attributed to global warming or El Nino have resulted in major crop damage, and the rice shortages caused by severe cold-weather damage in 1993 are still remembered. Culinary plants include many crops of tropical origin among fruits and vegetables such as tomatoes, cucumbers, melons, and water melon. Such crops are in high demand and are extremely important in terms of agriculture, and they have long been involved in greenhouse culture. However, since the oil shock of 1974, the conservation of resources and lowering warming costs have become a problem. The conservation of resources in protected horticulture has been studied from a variety of perspectives, from the structural concerns of green houses to cultivation techniques, but the most basic consideration is increasing the cold tolerance of crops.

Hot temperature can be a major form of stress for plants, and, in particular, the growth and yield of crops is tremendously affected by heat during summer.

In regard to salt stress, it is said that about 10% of all land surface area is salt damaged, and the spread of saline soil, primarily in arid areas such as Southeast Asia and Africa is becoming a serious agricultural problem.

Drought stress can be a major form of stress for plants, and is significantly affected by the amount and distribution of precipitation when temperature is not a limiting factor. The growth and yield of crops is tremendously dependent upon drought stress in semi-arid regions and the like which are important areas of crop cultivation.

Osmotic stress or water stress can be a major form of stress for plants, and is significantly affected by the amount and distribution of precipitation when temperature is not a limiting factor. The growth and yield of crops is tremendously dependent upon osmotic stress or water stress in semi-arid regions and the like which are important areas of crop cultivation.

Cross breeding, breeding making use of recent genetic engineering techniques, methods making use of the action of plant hormones and plant regulators, and the like have been employed to improve tolerance against these various types of stress.

Stress-tolerant plants have thus far been produced using genetic engineering techniques. Genes reported to have been used in the improvement of cold tolerance include fatty acid desaturase genes of membrane lipids (ω-3 desaturase gene, glycerol-3-phosphate acyltransferase gene, and stearoyl-ACP-desaturase gene), pyruvic-phosphate dikinase genes involved in photosynthesis, and genes coding for proteins with cryoprotection/prevention activity (COR15, COR85, and kin1).

Genes reported to have been used in the improvement of tolerance against salt, drought, and water stress include glycine betaine synthetase genes of osmotic regulators (choline monooxygenase gene and betaine aldehyde hydrogenase gene) and proline synthetase genes (1-pyroline-5-carboxylate synthetase).

As the method of improving the stress tolerance by increasing the expression amount of the stress defense gene involved in the stress tolerance or the stress resistance, the method of utilizing a gene encoding a transcription factor (DREB gene) has been reported (Non-patent document 1: Nature Biotechnology, 17, 287-291, 1999; Patent document 2: The Plant Cell, 10, 1391-1406, 1998). In both reports, it has been described that the expression amount of the stress defense gene group such as rd29A, kin1 and P5CS are increased to enhance the tolerance against drought stress, salt stress and freeze stress by excessively expressing the DREB gene constantly in plants, but remarkable inhibition of growth is observed, individuals which stop the growth and development and are withered are observed, and adverse effects on the growth and development are shown.

There are the cases in which the gene involved in the stress tolerance or the stress resistance other than DREB gene has been introduced. It has been known that a cold regulated protein/LEA protein is a late embryogenesis abundant (LEA) protein and is induced by stress, and it has been reported that the tolerance against drought stress and salt stress is enhanced by introducing HV1 which is the LEA protein into rice plant (Plant Physiology, 110, 249-257, 1996). It has been reported that a pathogen related PR-1 protein is a protein induced by pathogen infection and that the tolerance against heavy metal stress and pathogen infection stress is enhanced by introducing a CABPR1 gene which is one of PR-1 (pathogenesis-related protein 1) into tobacco (Plant Cell Rep., Feb. 18, 2005). Peroxidase is known to be one (EC 1.11.1.7) of cell wall enzymes in the plants and to be induced by disease and stress. It has been reported that the tolerance against oxidative stress and pest stress is enhanced by introducing it into the plants (Plant Physiology, 132, 1177-1185, 2003; J. Econ. Entomol., 95(1), 81-88, 2002). However, for these stress tolerance and stress resistance, at most only two types of stress tolerances are imparted by introducing one type of the gene involved in the stress tolerance and the stress resistance into the plant. In the natural world, the plants suffer multiple stresses simultaneously, and thus, it is necessary to impart defense effects on the multiple stresses simultaneously in order to increase productivity of crops. Also, in many of the plants transformed with the forgoing genes, actually the sufficient effect to an industrially applicable extent has not been obtained, and actually these plants have not come into practical use.

There are genes suggested to be deeply involved in stress although they have not been introduced into the plant. It has been reported that an old regulated protein/cor15 is a gene induced by low temperature stress and is deeply involved in freeze stress tolerance (Pro. Natl. Acad. Sci. USA, 93, 13404-13409, 1996; Pro. Natl. Acad. Sci. USA, 95, 14570-14575, 1998). It has been reported that an early response dehydration protein/ERD15 is a gene induced by drought stress and is deeply involved in drought stress tolerance (Plant Physiology, 106, 1707, 1994). It has been reported that a salt stress induced tonoplast intrinsic protein/aquaporin and a water channel protein/aquaporin of Gene Number 7 are water channel proteins induced by stress, and are deeply involved in the tolerance against salt stress, osmotic stress and low temperature stress (Mol. Cells., 9(1), 84-90, 1999; Foods Food Ingredients J. Jpn., 176, 40-45). It has been reported that a dehydration induced protein/RD22 is a protein induced by drought stress, and is deeply involved in drought stress tolerance (Plant Cell., 15(1), 63-78, 2003). It has been reported that a senescence associated protein sen1 is a protein induced by aging stress, salt stress, osmotic stress and low temperature stress, and is deeply involved in the tolerance against aging stress, salt stress, osmotic stress and low temperature stress (Plant Physiology, 130, 2129-2141, 2002). Additionally, there are genes shown to be involved in the stress tolerance and the stress resistance. If these genes are introduced into the plant, the stress resistance corresponding to each gene is enhanced, but it is difficult to increase the expression amounts of the multiple stress defense genes simultaneously.

Owing to environmental problems and food problems, it is a very important subject to impart the stress defense effects to the plants, and several attempts to improve the stress defense have been performed by gene recombination technology, but actually the sufficient effect to an industrially applicable extent has not been obtained, and actually no plants has come into practical use. Furthermore, the expression levels of the stress defense gene group have been increased to improve the stress tolerance by excessively expressing the gene encoding DREB which is one of transcription factors in the plants. However, the remarkable inhibitory effect on the growth and development is observed, and it is problematic in that seeds can not be collected. Therefore, it is an object of the present invention to provide a method of imparting various stress defense effects by simply increasing expression amounts of multiple stress defense genes within the range in which no adverse effect is given to the growth and development of plants before and/or during encountering stress

### DISCLOSURE OF THE INVENTION

As a result of an extensive study for accomplishing the above object, the present inventors have found that a polyamine amount before and during encountering stress is increased by isolating a spermidine synthase (SPDS) gene, introducing the gene into a plant and excessively expressing the gene under promoter control, thereby increasing expression levels of multiple genes involved in stresses and improving parameters for various stress tolerances within the range in which no adverse effect is given to the growth and development of plants. Furthermore, the present inventors have found that various stress tolerance parameters are improved by similarly increasing the expression levels of the multiple genes involved in stresses to impart the stress defense effects using not only the spermidine synthase (SPDS) gene but also an S-adenosylmethionine decarboxylase (SAMDC) gene, an arginine decarboxylase (ADC) gene, an ornithine decarboxylase (ODC) gene and a spermine synthase (SPMS) gene capable of controlling an amount of contained spermidine or spermine. In addition, the present inventors have found that it can be important to excessively express in a form containing 5′-non-translated region concerning the ADC gene, the ODC gene and the SAMDC gene having 5′-non-translated region (e.g., uORF: small upstream open reading frame) which has been described to control an expression amount and a translation amount. That is, the present invention relates to imparting the stress defense effects to the plants.

1. A method of inducing an expression of at least two stress defense genes in a plant, characterized in that an expression amount of at least one stress defense gene is increased compared with a non-transformant by transforming the plant with an exogenous spermidine synthase (SPDS) gene, an exogenous S-adenosylmethionine decarboxylase (SAMDC) gene, an exogenous arginine decarboxylase (ADC) gene, an exogenous ornithine decarboxylase (ODC) gene and/or an exogenous spermine synthase (SPMS) gene under the control of a promoter capable of functioning in the plant.

16. A method of imparting stress defense effects to a plant characterized in that expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an ornithine decarboxylase (ODC) gene and/or a spermine synthase (SPMS) gene under the control of a promoter capable of functioning in the plant.

17. A method of imparting stress defense effects to a plant characterized in that expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an exogenous ornithine decarboxylase (ODC) gene and/or an exogenous spermine synthase (SPMS) gene under the control of a promoter capable of functioning in the plant, and a transformed plant in which expression levels of the stress defense genes have been increased compared with a non-transformed plant (wild type) is selected

18. A method of enhancing productivity of a plant characterized in that expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an exogenous ornithine decarboxylase (ODC) gene and/or an exogenous spermine synthase (SPMS) gene under the control of a promoter capable of functioning in the plant.

19. A method of enhancing stress tolerance in a plant characterized in that expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an exogenous ornithine decarboxylase (ODC) gene and/or an exogenous spermine synthase (SPMS) gene under the control of a promoter capable of functioning in the plant.

According to the present invention, disorders due to various stresses encountered in the growth, development and cultivation processes can be avoided, the growth inhibition and yield reduction can be reduced, as well as the stabilization of the cultivation, enhancement of productivity, enlargement of cultivation regions and expansion of cultivation periods can be anticipated. It becomes possible to cultivate the plants in barren lands and salt accumulated soils, and it can be anticipated to contribute to the global warming and the food problem.

### BRIEF DESCRIPTION OF THE DRAWINGS

FIG. 1 is a view showing results of Northern blotting of transformants (TSP, OSP) in which a polyamine synthase gene has been introduced and a wild type (WT).

FIG. 2 is a view comparing with respect to the tolerance against osmotic stress between transformants (TSP) in which a polyamine synthase gene has been introduced and a wild type (WT).

FIG. 3 is a view comparing with respect to the tolerance against drought stress between transformants (TSP) in which a polyamine synthase gene has been introduced and a wild type (WT).

FIG. 4 is a view comparing with respect to the tolerance against drought stress between transformants (OSP) in which a polyamine synthase gene has been introduced and a wild type (WT).

FIG. 5 is a view comparing with respect to the tolerance against cold stress between transformants (TSP) in which a polyamine synthase gene has been introduced and a wild type (WT).

FIG. 6 is a view comparing with respect to the tolerance against salt stress between transformants (TSP) in which a polyamine synthase gene has been introduced and a wild type (WT).

FIG. 7 is a view comparing with respect to the tolerance against salt stress between transformed sweet potato in which a polyamine synthase gene has been introduced and a wild type (WT).

FIG. 8 is a view comparing with respect to the formation rate of the root tubers between transformed sweet potato in which a polyamine synthase gene has been introduced and a wild type (WT).

FIG. 9 is a photograph comparing with respect to the root sight between transformed sweet potato in which a polyamine synthase gene has been introduced and a wild type (WT).

FIG. 10 is a view comparing with respect to the yield of the root tubers between transformed sweet potato in which a polyamine synthase gene has been introduced and a wild type (WT).

FIG. 11 is a photograph comparing with respect to the root sight between transformed sweet potato in which a polyamine synthase gene has been introduced and a wild type (WT).

FIG. 12 is a view comparing with respect to the free polyamine content in the leaf and the root tuber between transformed sweet potato in which a polyamine synthase gene has been introduced and a wild type (WT).

### DETAILED DESCRIPTION OF THE INVENTION

As used in the present invention, “stress” refers to stress received from the environment, such as high temperatures, low temperatures, low pH, low oxygen, oxidation, osmotic pressure, drought, water, weak light, cadmium, ozone, air pollution, UV rays, pathogens, salt, herbicides, intense light, flooding, aging, heavy metals and pests. As used in the present invention, “non-transformants” or “untransformed plants” mean any plants to which at least one gene selected from exogenous spermidine synthase (SPDS) genes, S-adenosylmethionine decarboxylase (SAMDC) genes, arginine decarboxylase (ADC) genes, ornithine decarboxylase (ODC) genes and spermine synthase (SPMS) genes has not been introduced. As such, wild species, as well as cultivated varieties established through common cross breeding, natural or artificial variants thereof, transgenic plants incorporating exogenous genes other than spermidine synthase genes, and the like are all included. Documents cited in this specification are incorporated herein by reference.

Polyamine Synthase Gene

In the present invention, unexpectedly it has been found that the expression amounts of multiple genes (stress defense genes) involved in stress defense are increased by introducing a polyamine synthase gene into the plant. Furthermore, the present inventors have found the method in which the expression amounts of the multiple stress defense genes are increased within the range in which no adverse effect is given to the growth and development of the plants to impart various stress defense effects by transforming the plants particularly with the polyamine synthase gene such as spermidine synthase (SPDS) gene, S-adenosylmethionine decarboxylase (SAMDC) gene, arginine decarboxylase (ADC) gene, ornithine decarboxylase (ODC) gene and spermine synthase (SPMS) gene capable of increasing the amount of contained spermidine or spermine among the polyamine synthase genes. Herein, the SPDS gene, the SAMDC gene, the ADC gene, the ODC gene and the SPMS gene are sometimes collectively referred to as the “polyamine synthase gene”. Therefore, the “polyamine synthase gene” includes the SAMDC gene, the ADC gene, the ODC gene and the SPMS gene.

As used in the present invention, “spermidine synthase” (SPDS) genes” are genes cording for amino acids of enzymes involved in spermidine or spermine biosynthesis. The SPDS is a rate-limiting enzyme which produces spermidine. Spermidine synthase (SPDS: EC2.5.1.16) is an enzyme catalyzing the reaction producing spermidine and methylthioadenosine from putrescine and adenosylmethylthiopropylamine. “Arginine decarboxylase (ADC) genes” are genes coding for amino acids of enzymes involved in spermidine or spermine biosynthesis. The ADC is a rate-limiting enzyme which produces spermidine. Arginine decarboxylase (ADC: EC4.1.1.19.) is an enzyme catalyzing the reaction producing agmatine and carbon dioxide from L-arginine. “S-adenosylmethionine decarboxylase (SAMDC) genes” are genes coding for amino acids of enzymes involved in spermidine or spermine biosynthesis. S-adenosylmethionine decarboxylase (SAMDC: EC4.1.1.50.) is an enzyme catalyzing the reaction producing adenosylmethylthiopropylamine and carbon dioxide from S-adenosylmethionine. “Ornithine decarboxylase (ODC) genes” are genes coding for amino acids of enzymes involved in spermidine or spermine biosynthesis. Ornithine decarboxylase (ODC: EC4.1.1.17.) is an enzyme catalyzing the reaction producing putrescine and carbon dioxide from L-ornithine. “Spermine synthase (SPMS) genes” are genes coding for amino acids of enzymes involved in spermine biosynthesis. The SPMS is a rate-limiting enzyme which produces spermidine. For carrying out the present invention, the most preferable “polyamine synthase genes” are SPDS genes.

These polyamine synthase genes may be derived from any, such as plants, microbes and animals, as long as the gene expression for stress defense is enhanced without causing adverse effects on growth. Genes which are already isolated can also be used. For example, spermidine synthase (SPDS) genes have been isolated from plants such as Arabidopsis thaliana and tabacco (Plant Cell Physiol., 39(1), 73-79, (1998)), tomatoes (Plant Physiol., 120, 935, (1999)), coffee (Plant Science., 140, 161-168, (1999)), peas (Plant Molecular Biology, 39, 933-943, (1999)), apples (Mol. Gen. Genomics, 268, 799-807, (2003)), Cucurbita ficifolia Bouche (WO02/23974, WO03/84314). Furthermore, it has been attempted that the spermidine synthase (SPDS) gene is introduced into tobacco which is a model plant, and the change of contained polyamine has been examined, but the expression level of the stress defense genes and the improvement of environmental stress tolerances have not been examined (Journal of Plant Physiology, 162: 989-1001, 2004). Arginine decarboxylase (ADC) genes have been isolated from oats (Mol. Gen. Genet., 224, 431-436 (1990)), tomatoes (Plant Physiol., 103, 829-834 (1993)), Arabidopsis thaliana (Plant Physiol., 111, 1077-1083 (1996)), peas (Plant Mol. Biol., 28, 997-1009 (1995)), and Cucurbita ficifolia Bouche (WO02/23974, WO03/84314); S-adenosylmethionine decarboxylase (SAMDC) genes have been isolated from potatoes (Plant Mol. Biol., 26, 327-338 (1994)), spinach (Plant Physiol., 107, 1461-1462 (1995)), and Cucurbita ficifolia Bouche (WO02/23974, WO03/84314); ornithine decarboxylase (ODC) genes have been isolated from datura (Biochem. J., 314, 241-248 (1996)); spermine synthase (SPMS) genes have been isolated from Arabidopsis thaliana (EMBO J., 19, 4248-4256, (2000))

In one preferable embodiment of the present invention, it is important that the ADC gene, the ODC gene or the SAMDC gene is excessively expressed in a form of containing the 5′-non-translated region (e.g., uORF) which affects the expression and the translation under the control of an inducible type promoter.

The “uORF” of the present invention indicates an upstream open reading frame, and is present 5′ upstream (5′-non-translated region) of the ORF which encodes amino acids. The uORF is present in the 5′-non-translated region of the polyamine synthase genes (ADC gene, ODC gene and SAMDC gene), and is believed to control the expression and the translation of the polyamine synthase genes.

According to the present invention, the polyamine synthase gene isolated from microorganisms and animals can express the stress defense genes such as DREB, CBF1, LEA and COR about 1.1 to 10 times, preferably about 1.3 to 10 times, more preferably about 1.4 to 8 times and particularly about 1.5 to 5 times more highly compared with non-transformants. More preferably, the polyamine synthase gene isolated from the microorganisms and the animals can express the stress defense genes such as DREB, CBF1, LEA and COR in the range of about 1.5 to 4 times more highly compared with the non-transformants.

In addition, polyamine synthase genes can also be isolated from various plants. Specific examples include dicotyledons such as Cucurbitaceae; Solanaceae; Brassicaceae such as Arabidopsis thaliana; Papilionaceae such as alfalfa and Vigna unguiculata; Malvaceae; and Asteraceae; or monocotyledons such as Gramineae, including rice, wheat, barley, and corn. Cucurbitaceae, Brassicaceae and Gramineae are preferred, and Cucurbita ficifolia Bouche, Arabidopsis thaliana, rice, corn, wheat, cotton, soybeans and rapeseed are more preferred.

In the invention, the most suitable conditions for obtaining polyamine synthase genes are also found. That is, plant tissue in which the plant-derived polyamine synthase genes of the invention are isolated may be in the form of seeds or in the process of growing. The genes may be isolated from part or all of the tissue of growing plants. Any part can be used to isolate genes, but whole plants, buds, flowers, ovaries, fruit, leaves, stems, roots, and the like are preferred. Roots and leaves are more preferred.

Preferred examples of polyamine metabolism-related enzyme genes used in the present invention include the spermidine synthase gene. Specific examples include:

DNA having the base sequence represented by base numbers 77 through 1060 in the base sequence given in SEQ ID NO. 1 (Cucurbita ficifolia Bouche);

DNA having the base sequence represented by base numbers 118 through 1281 in the base sequence given in SEQ ID NO. 3 (rice); and

DNA having the base sequence represented by base numbers 456 through 1547 in the base sequence given in SEQ ID NO. 5 (Cucurbita ficifolia Bouche)

DNA having the base sequence represented by base numbers 541 through 2661 in the base sequence given in SEQ ID NO. 7 (Cucurbita ficifolia Bouche).

DNA having the base sequence represented by base numbers 1 through 1020 in the base sequence given in SEQ ID NO. 9 (Arabidopsis thaliana)

Further examples include:

DNA having a base sequence capable of hybridizing under stringent conditions with DNA or their complementary chains having any of the above sequences, and coding for a polypeptide with spermidine synthase activity equivalent to those sequences.

Still further examples include:

DNA comprising any of the above amino acid sequences with 1 or more bases deleted, substituted, inserted, or added, and coding for a polypeptide with spermidine synthase activity equivalent to those sequences.

“Polyamine synthase genes” include known genes as well as DNA having a base sequence capable of hybridizing under stringent conditions with the genes or their complementary chains, and coding for a polypeptide with polyamine synthase activity equivalent to those sequences. Furthermore, DNA comprising amino acid sequences encoded by any of the above DNA, in which 1 or more bases deleted, substituted, inserted, or added, and coding for a polypeptide with polyamine synthase activity equivalent to those sequences are included.

The “stringent conditions” referred to here mean conditions under which base sequences coding for a polypeptide with enzyme activity equivalent to the polyamine synthase (e.g. spermidine synthase) encoded by a specific polyamine synthase gene (e.g. polyamine synthase genes) sequence form hybrids with the specific sequence (referred to as specific hybrids), and base sequences coding for polypeptides with no such equivalent activity do not form hybrids with the specific sequence (referred to as non-specific hybrids). One with ordinary skill in the art can readily select such conditions by varying the temperature during the hybridization reaction and washing process, the salt concentration during the hybridization reaction and washing process, and so forth. Specific examples include, but are not limited to, conditions under which hybridization is brought about at 42° C. in 6×SSC (0.9 M NaCl, 0.09 M trisodium citrate) or 6×SSPE (3M NaCl, 0, 2 M NaH₂PO₄, 20 mM EDTA-2Na, pH 7.4), and the product is washed with 0.5×SSC at 42° C. Preferably, the condition where the hybridization is performed in 50% formaldehyde, 6×SCC (0.9 M NaCl, 0.09M trisodium citrate) or 6×SSPE (3 M NaCl, 0.2 M NaH₂PO₄, 20 mM EDTA2Na, pH 7.4) at 42° C. and washing with 0.1×SCC at 42° C. is further performed is included.

The “base sequences with 1 or more bases deleted, substituted, inserted, or added” referred to here are widely known by those having ordinary skill in the art to sometimes retain physiological activity even when the amino acid sequence of a protein generally having that physiological activity has one or more amino acids substituted, deleted, inserted, or added. Genes that have such modifications and that code for a polyamine synthase (e.g. spermidine synthase) can also be used in the present invention. For example, the poly A tail or 5′,3′ end nontranslation regions may be “deleted,” and bases may be “deleted” to the extent that amino acids are deleted. Bases may also be “substituted,” as long as no frame shift results. Bases may also be “added” to the extent that amino acids are added. However, it is essential that such modifications do not result in the loss of polyamine synthase (e.g. spermidine synthase) activity. “Genes with one or more bases deleted, substituted, or added” are preferred. Such modified DNA can be obtained by modifying the DNA base sequences of the invention so that amino acids at specific sites are substituted, deleted, inserted, or added by site-specific mutagenesis, for example (Nucleic Acid Research, Vol. 10, No. 20, 6487-6500 (1982)).

Polyamines

Polyamines, the general term for aliphatic hydrocarbons with 2 or more primary amino groups, are ubiquitous natural substances in organisms, with more than 20 types discovered so far. Typical polyamines include putrescine, spermidine, and spermine. The known primary physiological action of polyamines includes (1) nucleic acid stabilization and structural modification through interaction with nucleic acids; (2) promotion of various nucleic acid synthesis systems; (3) activation of protein synthesis systems; and (4) stabilization of cell membranes and enhancement of membrane permeability of substances. Reports on the role of polyamines in plants include cell protection and promotion of nucleic acid or protein biosynthesis during cellular growth or division. As used in the invention, “spermidine”, one of the typical polyamines, is an ubiquitous natural substance in organisms, and is an aliphatic hydrocarbon with three primary amino group.

The involvement of polyamines in various types of environmental stress has recently been reported. They have been implicated in cold stress (J. Japan Soc. Hortic. Sci., 68, 780-787 (1999); J. Japan Soc. Hortic. Sci., 68, 967-973 (1999); Plant Physiol. 124, 431-439 (2000)); salt stress (Plant Physiol. 91, 500-504 (1984)); acid stress (Plant Cell Physiol. 38(10), 1156-1166 (1997)); osmotic stress (Plant Physiol. 75, 102-109 (1984)); pathogen infection stress (New Phytol., 135, 467-473 (1997)); and herbicide stress (Plant Cell Physiol. 39(9), 987-992 (1998)), but all of these reports assume the involvement of polyamines based on the correlation between growth reaction or stress tolerance and changes in polyamine concentration, yet their involvement at the genetic level between environmental stress tolerance and polyamine synthase genes was not well studied.

There are other cases in which the polyamine synthase gene has been introduced into the plant, but regulation of the expression amount of the stress defense gene has not been studied. For example, the plant transformed with the spermidine synthase (SPDS) gene has been reported in tobacco (Non-patent document 16: Journal of Plant Physiology 161, 989-1001, 2004). The SPDS gene derived from Datura stramonium has been excessively expressed constantly in tobacco, and the change of contained polyamine amounts has been examined. However, the change of the expression amount of the stress defense genes and the stress tolerance are not shown at all. The method of increasing the expression amounts of the stress defense gene group by transforming the plant with the spermidine synthase (SPDS) gene thereby imparting the stress defense effects has not been reported until now. Also, the method of increasing the expression amounts of the stress defense gene group by transforming the plant with the S-adenosylmethionine decarboxylase (SAMDC) gene, the arginine decarboxylase (ADC) gene, the ornithine decarboxylase (ODC) gene and/or the spermine synthase (SPMS) gene thereby imparting the stress defense effects has not been reported until now. In addition, it has been attempted that the polyamine synthase gene is introduced into the plant, and the change of the contained polyamine amounts has been examined, but the relationship between the expression level of the stress defense gene and the improvement of various stress tolerances has not been examined at all. In the present invention, the relationship between the expression levels of the stress defense genes and stress defense genes and the improvement of various stress tolerances has been disclosed for the first time.

As a result of an extensive study for imparting the stress defense effects to the plants, the present inventors have found that it is very important for imparting or improving various stress tolerances to increase the amount of contained spermidine or spermine before or during encountering stresses by transforming the plant with the spermidine synthase (SPDS) gene and increase the expression levels of the stress defense genes by the action of increased spermidine or spermine thereby imparting the stress defense effects. Without wishing to be bound to any theory, the present inventors believe that (1) spermidine or spermine increased by transforming the plant with the SPDS gene acts as a second messenger (signal transducing substance) and activates tyrosine kinase involved in signal transduction thereby inducing the expression of the stress defense genes, and (2) spermidine or spermine increased by transforming the plant with the SPDS gene is metabolized by polyamine oxidase (PAO) resulting in increased levels of hydrogen peroxide which activate the signal transduction thereby inducing the expression of the stress defense genes. Since the increase of contained spermidine or spermine and the action thereof are important for the induction of the expression of the stress defense genes and the increase of the expression level thereof, likewise the effect of increasing the expression levels of the stress defense genes is obtained also using the S-adenosylmethionine decarboxylase (SAMDC) gene, the arginine decarboxylase (ADC) gene, the ornithine decarboxylase (ODC) gene or the spermine synthase (SPMS) which can increase the amount of contained spermidine or spermine. A time period that the expression level of the stress defense gene is enhanced may be any of constantly, under the condition of non-stress, before encountering stress and under the condition of stress. However, the present inventors have found that it is important to increase the expression amounts of the stress defense genes within the range in which no effect is given to the growth and development of the plant constantly or under the condition of non-stress and impart the stress defense effects to the plant previously (preliminarily) before encountering stress (vaccine effect), thereby exhibiting the excellent tolerance and resistance against various stresses when encountering stress. In addition, the present inventors have found that the expression levels of the stress defense genes such as DREB, CBF1 and COR are increased within the range in which no adverse effect is given to the growth and development to impart the stress defense effects by introducing the gene such as SPDS, SAMDC, ADC, ODC and SPMS into the plant and excessively expressing it under the control of the promoter, thereby improving the parameters of various stress tolerances and enhancing the productivity (e.g., yield) and characters, and have completed the present invention.

Stress Defense Genes

In the present invention, the “stress defense gene” is a gene whose expression/induction or expression amount is increased when the plant encounters stress, and the gene involved in or associated with the stress tolerance. In one preferable embodiment of the invention, the stress defense genes are the following 49 genes or the genes having 60% or more, preferably 70% or more, more preferably 80% or more, still more preferably 85% or more and particularly 90% or more homology to these genes with specific Accession Number.

TABLE 1

| Gene | | Accession |
|---|---|---|
| number | Stress defense gene | Number |
| 1 | transcription factor/CBF1, DREB1B | AT4G25490 |
| 2 | cold regulated protein/LEA protein | AT2G03740 |
| 3 | cold regulated protein/cor15 | AT2G42530 |
| 4 | pathogen related PR-1 protein/PR-1 | AT2G14610 |
| 5 | early response dehydration protein/ERD15 | D30719 |
| 6 | salt stress induced tonoplast intrinsic protein/ | AF004393 |
| | aquaporin | |
| 7 | water channel protein/aquaporin | AAC79629 |
| 8 | dehydration induced protein/RD22, rd22 | AT5G25610 |
| 9 | stress responsive protein | CAB52439 |
| 10 | drought induced protein | AT1G72290 |
| 11 | low temperature and salt responsive protein | CAB79783 |
| 12 | stress responsive protein | CAB52439 |
| 13 | zinc finger protein | AT5G43170 |
| 14 | disease resistance protein | BAB08633 |
| 15 | disease resistance protein | AT3G05660 |
| 16 | disease resistance protein | BAB09430 |
| 17 | disease resistance protein | AT5G18350 |
| 18 | disease resistance protein | AT4G11210 |
| 19 | Peroxidase | AT4G33420 |
| 20 | senescence associated protein sen1 | AT4G35770 |
| 21 | senescence associated protein | BAB33421 |
| 22 | nematode resistance protein/Hs1pro-1 | NP181529 |
| 23 | WRKY transcription factor | AT4G23810 |
| 24 | RMA1 RING zinc finger protein | BAA28598 |
| 25 | transcriptional activator CBF1/CBF1 | AT1G12630 |
| 26 | zinc finger protein | AT3G07650 |
| 27 | transcription factor/DREB1A | AT1G63030 |
| 28 | transcription factor/DREB1A | AT1G63040 |
| 29 | stress induced protein sti1 | T48150 |
| 30 | early response dehydration protein/ERD15 | T02438 |
| 31 | B-box zinc finger protein | AT1G68520 |
| 32 | transcription factor/DREB2B | AT3G11020 |
| 33 | heat shock protein DnaJ homolog | AT4G36040 |
| 34 | pathogenesis related protein | T04989 |
| 35 | myb protein | AT4G372760 |
| 36 | jasmonic acid regulatory protein | AAF35416 |
| 37 | early response dehydration protein/ERD15 | D30719.1 |
| 38 | low temperature induced protein 78/LT178, | AT5G52310 |
| | rd29A, COR78 | |
| 39 | salt tolerance zinc finger protein | CAA64820 |
| 40 | CCCH type zinc finger protein | AT2G25900 |
| 41 | Cytochrome P450 | AT5G45340 |
| 42 | transcription activator CBF1/CBF1 | AT1G12610 |
| 43 | DREB like AP2 domain transcription | AT2G38340 |
| | factor/DREB2E | |
| 44 | Peroxidase | AT5G64120 |
| 45 | AP2 domain protein | AT1G78080 |
| 46 | senescence associated protein sen1 | AT4G35770 |
| 47 | stress responsive protein | CAB52439 |
| 48 | zinc finger protein | AT5G43170 |
| 49 | disease resistance protein | BAB08633 |

In another preferable embodiment, the stress defense genes can belong to the following 1 to 11 categories:

I. CBF1/DREB1B

For example, CBF1, DREB1B of Gene Numbers 1, 25 and 42 are the genes encoding the transcription factors expressed and induced by stresses (The Plant Cell, 10, 1391-1406, 1998, Proc. Natl. Acad. Sci. U.S.A., 94, 1035-1040, 1997, Plant Physiol., 130, 639-648, 2002), and are shown to have an ERF/AP2 DNA binding domain and act as a factor to activate the transcription (Biochem. Biophys. Res. Commun., 290, 998-1009, 2002, Physiol. Plant, 112, 171-175, 2001). It has been reported that the tolerance against environmental stresses such as drought, freeze and low temperature is enhanced by introducing CBF1, DREB1B genes into the plant (Science, 280, 104-106, 1998, Plant Physiol., 127, 910-917, 2001, Plant Physiol., 130, 618-626, 2002).

II. CBF3/DREB1A

For example, DREB1A, CBF3 of Gene Numbers 27 and 28 are the genes encoding the transcription factors expressed and induced by stresses (The Plant Cell, 10, 1391-1406, 1998, Plant J., 16, 433-442, 1998, Biochem. Biophys. Res. Commun., 250, 161-170, 1998, Plant Physiol., 130, 639-648, 2002), and are shown to have an ERF/AP2 DNA binding domain and act as a factor to activate the transcription (Plant Cell, 13, 61-72, 2001, Biochem. Biophys. Res. Commun., 290, 998-1009, 2002, The Plant Journal, 38, 982-993, 2004). It has been reported that the tolerance against environmental stresses such as drought, salt, freeze and low temperature is enhanced by introducing CBF1, DREB1B genes into the plant (The Plant Cell, 10, 1391-1406, 1998, Nat. Biotech., 17, 287-291, 1999, Plant Physiol., 124, 1854-1865, 2000)

III. DREB2B

For example, DREB2B of Gene Number 32 is the gene encoding the transcription factors expressed and induced by drought and salt stress (The Plant Cell, 10, 1391-1406, 1998, Plant Mol. Biol., 42, 657-665, 2000), and are shown to have an ERF/AP2 DNA binding domain and act as a factor to activate the transcription (Biochem. Biophys. Res. Commun., 290, 998-1009, 2002)

IV. LTI78, COR78, rd29A

For example, it has been reported that LTI78(low temperature induced protein 78), COR78 and rd29A of Gene Number 38 are the genes encoding the proteins expressed and induced by low temperature stress, drought stress and salt stress and are deeply involved in the tolerance against low temperature stress, drought stress and salt stress (Plant Physiol., 103, 1047-1053, 1993, Plant Mol. Biol., 21, 641-653, 1993, Plant Cell, 6,251-264, 1994, Journal of Experimental Botany, 47, 291-305, 1996, Plant Physiol, 130, 2129-2141, 2002). It has been reported that the genes (DREB1A, DREB2A) encoding the transcription factors which bind to the promoter of rd29A are isolated and that the expression amount of rd29A is increased by introducing the DREB1A gene into the plant thereby enhancing the tolerance against environmental stresses such as drought, salt, freeze and low temperature (The Plant Cell, 10, 1391-1406, 1998, Nat. Biotech., 17, 287-291, 1999, Plant Physiol., 124, 1854-1865, 2000).

V. RD22/rd22

For example, it has been reported that RD22 and rd22 of Gene Number 8 are the genes encoding the proteins expressed and induced by drought stress and are deeply involved in the tolerance against drought stress (The Plant Cell, 5, 1529-1539, 1993, Mol. Gen. Genet., 247, 391-398, 1995, The Plant Cell, 9, 1859-1868, 1997, Plant Cell., 15(1), 63-78, 2003)

VI. Cor15

For example, it has been reported that cor15 of Gene Number 3 is the gene encoding the protein expressed and induced by low temperature stress and drought stress and is deeply involved in the freeze stress tolerance (Plant Mol. Biol., 23, 1073-1077, 1993, Journal of Experimental Botany, 47, 291-305, 1996). It has been reported that the freeze stress tolerance is enhanced by introducing cor15 gene into the plant (Pro. Natl. Acad. Sci. USA, 93, 13404-13409, 1996, Journal of Plant Physiology, 163, 213-219, 2006), and that the freeze stress tolerance of a chloroplast was enhanced when the freeze stress tolerance of the chloroplast isolated from the transformed plant was examined (Pro. Natl. Acad. Sci. USA, 95, 14570-14575, 1998).

VII. ERD15

For example, it has been reported that ERD15 of Gene Number 5, 30 and 37 are the genes encoding the proteins induced by drought stress and are deeply involved in the tolerance against drought stress (Plant Physiology, 106, 1707, 1994)

VIII. LEA Protein

For example, LEA protein of Gene Number 2 is known to be the gene encoding late embryogenesis abundant (LEA) protein and to be expressed and induced by various stresses, and it has been reported that the tolerance against drought stress and salt stress is enhanced by introducing HVA1 gene which is the LEA protein gene derived from barley into rice plant, and that the salt stress tolerance is enhanced in yeast in which the LEA protein gene derived from tomato has been highly expressed (Plant Physiology, 110, 249-257, 1996, Gene, 170, 243-248, 1996).

IX. PR-1

For example, PR-1 of Gene Number 4 is known to be the gene encoding the protein induced by pathogen infection, and to have an antibacterial activity (Physiol. Mol. Plant Pathol., 55, 85-97, 1999). It has been reported that the tolerance against heavy metal stress and pathogen infection stress is enhanced by introducing CABPR1 (Capsicum annuum basic pathogenesis-related protein 1) gene which is one of PR-1 (pathogenesis-related protein 1) into tobacco (Plant Cell Rep., Feb. 18, 2005).

X. Peroxidase

For example, peroxidase of Gene Number 19 or 44 is known to be one (EC 1.11.1.7) of cell wall enzymes in the plant and to be induced by diseases and stresses, and it has been reported that the tolerance against oxidative stressoxidative stress and pest stress is enhanced by introducing it into the plant (Plant Physiology, 132, 1177-1185, 2003, J. Econ. Entomol., 95(1), 81-88, 2002).

XI. Hs1pro-1

For example, it has been reported that Hs1pro-1 (nematode resistance protein) gene of Gene Number 22 is isolated as a tolerance gene of nematode and is deeply involved in nematode tolerance (Science, 275, 832-834, 1997, Plant Mol. Biol., 52, 643-660, 2003).

In the above 49 genes or genes having 60% or more homology to the genes and further the genes belonging to I to XI, the description of the gene of each Gene Number is only an exemplification, and the stress defense gene can be changed depending on the type of the plant. Therefore, when the expression amounts of two or more of 49 genes, genes having 60% or more homology to the genes and the stress defense genes belonging to I to XI are increased in the transformant obtained by introducing the specific polyamine synthase gene into the plant, this case is included in the method of the present invention.

For the above stress defense genes, the expression amounts of two or more of 49 genes (including the genes having 60% or more homology) and distinct genes of I to XI may be increased, and the expression amounts of two or more of the genes belonging to the same group may be increased.

Imparting of Stress Defense Effects

As noted above, in the present invention, “stress” includes stress received from the environment, such as high temperatures, low temperatures, low pH, low oxygen, oxidation, salt, osmotic, drought, water, flooding, cadmium, copper ozone, air pollution, UV rays, intense light, weak light, pathogens, disease pests, herbicides and aging. Of these, “heat stress” is stress on plants due to exposure of the plants to environments over the upper limit of optimal growth temperature of the plant. Plants subject to heat stress are damaged as a result of gradual or sudden loss of cellular physiological function. “Cold stress” is stress on plants due to exposure of the plants to environments below the minimum optimal growth temperature of the plant. Plants subject to cold stress are damaged as a result of gradual or sudden loss of cellular physiological function. “Salt stress” is stress on plants due to exposure of the plants to environments over the maximum optimal growth salt concentration of the plant. Plants subject to salt stress are damaged as a result of gradual or sudden loss of cellular physiological function due to intracellular infiltration of excess salt. “Osmotic stress” is stress on plants due to exposure of the plants to environments over or under the maximum or minimum optimal growth osmotic of the plant. Plants subject to osmotic stress are damaged as a result of gradual or sudden loss of cellular physiological function. “Drought stress” is stress on plants due to exposure of the plants to environments under the minimum optimal growth moisture concentration of the plant. Plants subject to drought stress are damaged as a result of gradual or sudden loss of cellular physiological function. “Water stress” is stress on plants due to exposure of the plants to environments under the minimum optimal growth moisture concentration of the plant. Plants subject to water stress are damaged as a result of gradual or sudden loss of cellular physiological function. Stress under weak light is stress on plants due to exposure of the plants to environments under the minimum optimal growth light intensity of the plant. Plants subject to stress under weak light are damaged as a result of gradual or sudden loss of cellular physiological function. “Herbicide stress” is stress on plants due to exposure of the plants to environments over the maximum optimal growth herbicide concentration of the plant. Plants subject to herbicide stress are damaged as a result of gradual or sudden loss of cellular physiological function. “Pathogen stress” is stress which the plant receives by being infected or diseased with a pathogen which is unsuitable for plant growth, and the plant which has received pathogen stress damages physiological functions gradually or rapidly to cause the disease. “Pest stress” is stress which the plant receives by insect damage or being infected with or encountering a pest which is unsuitable for the plant growth, and the plant which has received pest stress damages physiological functions gradually or rapidly to cause the disease. “Heavy metal stress” is stress on plants due to exposure of the plants to environments under the minimum optimal growth heavy metal concentration of the plant. Plants subject to drought stress are damaged as a result of gradual or sudden loss of cellular physiological function.

In the present invention, the “method of imparting the stress defense effects” refers to the method of imparting the stress defense effects in comparison with the period before introduction by introducing the exogenous polyamine synthase gene into the plant. Specifically, the “method of imparting low temperature stress defense effects” is the method capable of avoiding or reducing the growth inhibition, the disease and the productivity decrease due to low temperature stress which the plant encounters in the growth process by imparting the stress defense effects to the plant. The “method of imparting high temperature stress defense effects” is the method capable of avoiding or reducing the growth inhibition, the disease and the productivity decrease due to high temperature stress which the plant encounters in the growth process by imparting the stress defense effects to the plant. The “method of imparting salt stress defense effects” is the method capable of avoiding or reducing the growth inhibition, the disease and the productivity decrease due to salt stress which the plant encounters in the growth process by imparting the stress defense effects to the plant. The “method of imparting osmotic stress defense effects” is the method capable of avoiding or reducing the growth inhibition, the disease and the productivity decrease due to osmotic stress which the plant encounters in the growth process by imparting the stress defense effects to the plant. The “method of imparting oxidative stressoxidative stress defense effects” is the method capable of avoiding or reducing the growth inhibition, the disease and the productivity decrease due to oxidative stressoxidative stress which the plant encounters in the growth process by imparting the stress defense effects to the plant. The “method of imparting herbicide stress defense effects” is the method capable of avoiding or reducing the growth inhibition, the disease and the productivity decrease due to herbicide stress which the plant encounters in the growth process by imparting the stress defense effects to the plant. The “method of imparting freeze stress defense effects” is the method capable of avoiding or reducing the growth inhibition, the disease and the productivity decrease due to freeze stress which the plant encounters in the growth process by imparting the stress defense effects to the plant. The “method of imparting drought stress defense effects” is the method capable of avoiding or reducing the growth inhibition, the disease and the productivity decrease due to drought stress which the plant encounters in the growth process by imparting the stress defense effects to the plant. The “method of imparting pathogen infection stress defense effects” is the method capable of avoiding or reducing the growth inhibition, the disease and the productivity decrease due to pathogen infection stress which the plant encounters in the growth process by imparting the stress defense effects to the plant. The “method of imparting pest stress defense effects” is the method capable of avoiding or reducing the growth inhibition, the disease and the productivity decrease due to pest stress which the plant encounters in the growth process by imparting the stress defense effects to the plant. The “method of imparting disease stress defense effects” is the method capable of avoiding or reducing the growth inhibition, the disease and the productivity decrease due to disease stress which the plant encounters in the growth process by imparting the stress defense effects to the plant. The “method of imparting aging stress defense effects” is the method capable of avoiding or reducing the growth inhibition, the disease and the productivity decrease due to aging stress which the plant encounters in the growth process by imparting the stress defense effects to the plant. The “method of imparting heavy metal stress defense effects” is the method capable of avoiding or reducing the growth inhibition, the disease and the productivity decrease due to heavy metal stress which the plant encounters in the growth process by imparting the stress defense effects to the plant. The stabilization of cultivation, the enhancement of productivity and yield, and the effective utilization of the cultivation, the environment, the period, the region and the area can be anticipated by using the expression amounts of the stress defense genes as indicators or controlling them. In addition, it is possible to anticipate the enhancement of productivity of various useful substances (e.g., starch, protein) obtained from the plants by increasing the productivity and the yield of the plants. The expression amount of the stress defense gene can be utilized as the indicator for the method of selecting and diagnosing cultivation effective crops.

A method of the invention can be carried out by introducing an exogenous polyamine synthase gene to plants having no exogenous polyamine synthase gene through genetic engineering means and making it retained in a stable manner. As used herein, “retained in a stable manner” means that the polyamine synthase gene is expressed in the plant at least in which the polyamine synthase gene has been introduced, and is retained in the plant cells long enough to result in the improvement of stress tolerance. The polyamine synthase gene is, therefore, preferably incorporated on the chromosomes of the host plant. The polyamine synthase gene or genes should even more preferably be retained in subsequent generations.

As used herein, “exogenous” means not intrinsic to the plant, but externally introduced. Accordingly, an “exogenous polyamine metabolism-related enzyme gene” may be a polyamine synthase gene homologous to the host plant (that is, derived from the host plant), which is externally introduced by genetic manipulation. The use of a host-derived polyamine synthase gene is preferred in consideration of the identity of the codon usage.

The exogenous polyamine synthase gene may be introduced into plants by any method of genetic engineering. Examples include protoplast fusion with heterologous plant cells having the polyamine synthase gene, infection with a plant virus having a viral genome genetically manipulated to express the polyamine synthase gene, or transformation of host plant cells using an expression vector containing the polyamine synthase gene.

The plants of the invention are preferably transgenic plants which are obtained by the transformation of cells of plants lacking the exogenous polyamine synthase gene in an expression vector containing the exogenous polyamine synthase under the control of a promoter capable of functioning in plants.

Examples of promoters capable of functioning in plants include the 35S promoter of the cauliflower mosaic virus (CaMV) which functions in plant cells, the nopaline synthase gene (NOS) promoter, octopine synthase gene (OCS) promoter, phenylalanine ammonia lyase (PAL) gene promoter, and chalcone synthase (CHS) gene promoter, ubiquitin (Ubi-1) promoter, peroxidase gene promoter. Other well-known plant promoters not limited to these are also available.

Constitutive promoters include a CaMV35S promoter, an actin promoter (Plant Cell, 2, 163-171, 1990), an ubiquitin promoter (Plant Mol. Biol., 18, 675-689, 1992) and a rice cyclophilin promoter (Plant Mol. Biol., 25, 837-843, 1994). If not only the promoter to constantly or constitutively express in entire organs but also the promoter specific for the organ or tissue is used, it is possible to express the objective gene only in the particular organ or tissue, and impart the stress defense effects only to the particular organ or tissue. As the promoter specific for leaf tissues, an aldP promoter (Mol. Gen. Genet., 248, 668-674, 1995) and a rbcs promoter (Plant Cell Physiol., 35, 773-778, 1994) can be utilized. As the promoter specific for flower tissues, a chsA chalcone synthase promoter (Plant Mol. Biol., 15, 95-109, 1990) and an LAT52 promoter (Mol. Gen. Genet., 217, 240-245, 1989) can be utilized. As the promoter specific for roots, root tubers and stem tubers, an SbPRP1 promoter (Plant Mol. Biol., 21, 109-119, 1993) and a sporamin promoter (Mol. Gen. Genet., 225, 369, 1991) can be utilized. For example, the stress defense effects can be imparted only to the root tuber by using the polyamine synthase gene and the sporamin promoter which works specifically for the root tuber.

As inducible promoters, a stress inducible promoter, a temperature inducible promoter, a light inducible promoter, a period inducible promoter, a pathogen inducible promoter, and a disease inducible promoter and the like can be utilized. For example, by the use of the polyamine synthase gene and the promoter (e.g., BN115 promoter: Plant physiol., 106, 917-928, 1999) which can induce the transcription only when the plant encounters the low temperature, it is possible to control polyamine metabolism of the plant only at low temperature to impart the low temperature stress defense effect. By the use of the polyamine synthase gene and the promoter (e.g., Atmyb2 promoter: The Plant Cell, 5, 1529-1539, 1993) which can induce the transcription only when the plant encounters the drought, it is possible to control polyamine metabolism of the plant only at drought to impart the drought stress defense effect. By the use of peroxidase promoter (U.S. Pat. No. 3,571,639, U.S. Pat. No. 3,259,178) induced by various stresses, it is possible to control polyamine metabolism of the plant only at various stresses to impart the various stress defense effect. Furthermore, by the use of the polyamine synthase gene and the promoter which works in a vegetative stage, it is possible to impart the stress defense effects only in the vegetative stage.

Preferably, from a notion that it is particularly important for the stress defense to previously (preliminarily) impart the stress defense effects to the plant (vaccine-like effect) by increasing the expression amounts of the stress defense genes within the range where no adverse effect is given to the growth and development of the plant before encountering stress, preferably the steady or constitutive promoter, the promoter specific for the organ or the tissue, and the promoter specific for the period depending on the growth and development can be used. In particular, the steady or constitutive promoter is preferable.

The exogenous polyamine synthase gene in the expression vector of the present invention is located downstream of the promoter so that transcription is controlled by the promoter capable of functioning in plants. A transcription termination signal (terminator region) capable of functioning in plants should also be added downstream of the polyamine synthase gene. An example is the terminator NOS (nopaline synthase) gene.

The expression vector of the present invention may also contain a cis-regulatory element such as an enhancer sequence. The expression vector may also contain a marker gene for selecting transformants such as a drug-resistance gene marker, examples of which include the neomycin phosphotransferase II (NPTII) gene, the phosphinothricin acetyl transferase (PAT) gene, and the glyophosate resistance gene. Because the incorporated gene is sometimes dropped in the absence of selection pressure, it is advantageous to ensure that a herbicide resistance gene is also present on the vector so that the use of a herbicide during cultivation will always result in conditions involving selection pressure.

To facilitate mass production and purification, the expression vector should also contain a selection marker gene (such as ampicillin resistance gene or tetracycline resistance gene) in E. coli and a replication origin capable of autonomous replication in E. coli. The expression vector of the present invention can be constructed in a simple manner by inserting the selection marker gene as needed and an expression cassette of the polyamine synthase gene at the cloning site of an E. coli vector (pUC or pBR series).

When the exogenous polyamine metabolism-related enzyme gene is introduced by infection with Agrobacterium tumefaciens or Agrobacterium rhizogenes, the polyamine synthase gene expression cassette can be inserted in the T-DNA region (region transferred to plant chromosome) on a Ti or Ri plasmid of the cells. At present, binary vector systems are used in standard methods of transformation with Agrobacterium. The necessary functions for T-DNA transfer are independently provided by both the T-DNA itself and the Ti (or Ri) plasmid, these structural elements being divided on separate vectors. The binary plasmid has 25 bp border sequences at both ends necessary for cleaving and combining the T-DNA, and the plant hormone gene inducing crown gall (or hairy root) is removed, simultaneously providing room for inserting the exogenous gene. Examples of commercially available binary vectors include pBI101 and pBI121 (by Clontech). The Vir region involved in the incorporation of the T-DNA has trans action on the separate Ti (or Ri) plasmid referred to as the helper plasmid.

Various conventionally known methods can be used for the transformation of the plants. Examples include the PEG method in which protoplasts are isolated from plant cells by treatment with a cell wall-degrading enzyme such as cellulase or hemicellulase, and polyethylene glycol is added to a suspension of the protoplasts and an expression vector containing the aforementioned polyamine synthase gene expression cassette to incorporate the expression vector into the protoplasts by a process such as endocytosis; the liposome method in which an expression vector is introduced by ultrasonic treatment or the like into lipid membrane vesicles such as phosphatidylcholine, and the vesicles are fused with protoplasts in the presence of PEG; methods of fusion in a similar process using micelles; and electroporation in which electrical pulses are applied to a suspension of protoplasts and an expression vector to incorporate the vectors in the external solution into the protoplasts. However, these methods are complicated in that they require a culturing technique for the redifferentiation of the protoplasts into plants. Processes for introducing the gene into intact cells with cell walls include direct injection such as microinjection in which a micropipette is inserted into cells to inject the vector DNA in the pipettes under hydraulic or gas pressure into the cells, or the particle gun method in which metal microparticles coated with DNA are accelerated through the detonation of an explosive or gas pressure and thus introduced into the cells, and methods involving the use of infection with Agrobacterium. Drawbacks of microinjection are the need for considerable training and the small number of cells that are handled. It is therefore more desirable to transform plants with more convenient methods such as the Agrobacterium method and the particle gun method. The particle gun method is useful in that genes can be directly introduced into the apical meristem of plants while cultivated. In the Agrobacterium method, the genomic DNA of a plant virus such as the tomato golden mosaic virus (TGMV) or another gemini virus is simultaneously inserted between the border sequences into the binary vector, so that the viral infection can spread throughout the entire plant and the target gene can be simultaneously introduced into the entire plant simply by inoculating cells at any location of the cultivated plant with the viral cell suspension. These methods are known in the art, and the ordinary skilled person can choose a suitable method for a plant being transformed.

Transformed plants in accordance with the invention can be evaluated, for example, for their cold stress tolerance by low temperature treatment for 1 to 10 days at 0 to 20° C., followed by growth at 25 to 30° C. to study the state of growth, low temperature damage, or the like. In the transformed plants produced in the present invention, for example, the low temperature stress tolerance can be evaluated by low temperature treatment at 0 to 20° C. for 1 to 10 days followed by growing at 25 to 30° C. to examine the state of growth, low temperature damages, and the like. The low temperature stress tolerance can be evaluated by growing maize at 10 to 18° C. for the whole growth period and examining the state of growth and the wet weight (yield). Heat stress tolerance can be evaluated by low temperature treatment for 1 to 10 days at 35 to 50° C., followed by growth at 25 to 30° C. to study the state of growth, high temperature damage, or the like. The high temperature stress tolerance can be evaluated by growing maize at 35 to 45° C. for the whole growth period and examining the state of growth and the wet weight (yield). Salt stress tolerance can be evaluated by studying the state of growth, salt stress damage, or the like following growth at 25 to 30° C. on medium containing 10 to 300 mM NaCl. The salt stress tolerance can be evaluated by growing the maize in the potting compost containing 10 to 15 mM NaCl for the whole growth period and examining the state of growth and the wet weight (yield). Drought and water stress tolerance can be evaluated by studying the state of growth and the extent of damage after the supply of water has been terminated. The tolerance against drought and water stress can be evaluated by growing the maize in the potting compost where watering is limited and examining the state of growth and the wet weight (yield).

Examples of plants which may be transformed in the invention include, but are not limited to, dicotyledons, monocotyledons, herbaceous plants, and shrubs. Examples include sweet potatoes, tomatoes, cucumbers, squash, melons, watermelon, tobacco, Arabidopsis thaliana, bell peppers, eggplant, beans, taro, spinach, carrots, strawberries, white potatoes, rice, corn, alfalfa, wheat, barley, soybeans, rapeseed, sorghum, Eucalyptus, poplar, kenaf, Eucommia ulmoides, sugarcane, sugar beet, cassaya, betterave, sago palm, Chenopodium album, lilies, orchids, carnations, roses, chrysanthemum, petunias, Torenia fournieri, antirrhinum, cyclamen, gypsohila, geranium, sunflowers, Zoisia japonica, cotton, matsutake mushrooms, shiitake mushrooms, mushrooms, ginseng, citrus fruits, bananas, and kiwi fruit. Sweet potatoes, tomatoes, cucumbers, rice, corn, soybeans, wheat, petunias, Torenia fournieri, Eucalyptus, and cotton are preferred.

According to the present invention, it became possible to increase the polyamine amount before or during encountering stress by introducing the polyamine synthase gene into the plant and to impart multiple stress defense effects by increasing the expression amounts of multiple genes involved in the stress tolerance or the stress resistance. Furthermore, it is possible to anticipate the stabilization of the cultivation, the enhancement of the productivity and the yield, and the enlargement of the cultivation regions and the areas. Furthermore, it is also possible to anticipate the enhancement of the productivity of useful substances (e.g., starch, natural dyes) obtained from the plants by increasing the productivity and the yield of the plants.

The plants of the present invention are not limited to entire plants (whole plants) and include callus thereof, seeds, all plant tissues, leaves, stems, vines, roots, root tubers or stem tubers, flowers and the like. In addition, progenies thereof are also included in the plants of the present invention.

### BEST MODE FOR CARRYING OUT THE INVENTION

The invention is illustrated in further detail by the following examples, but they are provided only as examples and do not in any way limit the scope of the invention.

Method of Analyzing Polyamine

A wild type (non-recombinant plant) and transformants (gene recombinant plants) can be simultaneously cultivated, and the amounts of polyamine contained in the leaf, stem, root, seed, fruit and the like can be examined. Since the amount of contained polyamine is changed depending on the state of growth, it is important to examine the tissues whose growth periods and cultivation periods are the same (e.g., in the case of leaves, the same leaf age) by cultivating under the same condition. Polyamine includes free polyamine, combined polyamine and bound polyamine, extraction methods thereof are different, but all can be analyzed (Plant Cell Physiol., 43(2), 196-206, 2002). As a specific example, the method of analyzing free polyamine in the leaf will be described in detail. The leaves (about 0.1 to 1.0 g) (young leaves at the same leaf age) are sampled and frozen/stored. Dilution internal standard solution (1,6-hexanediamine, internal standard content=7.5 or 12 nmol) and 5% perchloric acid aqueous solution (5 to 10 mL per 1.0 g specimen fresh weight) were added to the sampled specimen, and was thoroughly ground down and extracted using an omnimixer at room temperature. The ground solution was centrifuged at 4° C., 35,000×g for 20 minutes, and the supernatant was collected and was taken as the free polyamine solution. Four hundred microliters of free polyamine solution, 200 μL of saturated sodium carbonate aqueous solution, and 200 μL of dansyl chloride/acetone solution (10 mg/mL) were added into a microtube with a screw cap, and lightly mixed. After firmly closing with a tube stopper and covering with aluminum foil, dansylation was conducted by heating for 1 hour in a 60° C. water bath. After allowing the tube to cool, 200 μL of proline aqueous solution (100 mg/mL) was added and mixed. The tube was covered with aluminum foil and heated again for 30 minutes in a water bath. After standing to cool, the acetone was removed by spraying nitrogen gas, and then 600 μL of toluene was added and vigorously mixed. After allowing the tube to stand quietly and separate into 2 phases, 300 to 400 μL of toluene in the upper layer was separated into a microtube. The toluene was completely removed by spraying nitrogen gas. 100 to 200 μL of methanol was added to the tube and the dansylated free polyamine was dissolved. The free polyamine content of putrescine, spermidine and spermine was assayed by the internal standard method using high performance liquid chromatography connected to a fluorescence detector (exitation wavelength is 365 nm, emission wavelength is 510 nm). A μBondapak C18 (manufactured by Waters, Co.: 027324, 3.9×300 mm, particle size 10 μm) was used for the HPLC column. The polyamine content in the specimens was calculated by deriving the peak areas of the polyamine and internal standard from the HPLC charts of the standard solution and specimens. For example, from the results of the polyamine analysis, cell lines in which the amount of contained spermidine or spermine has been increased 1.1 to 4.0 times compared with non-transformed plant (wild type) which has not been transformed with the exogenous polyamine synthase gene are selected or screened in the cell lines of transformants transformed with the exogenous polyamine synthase gene.

Method of Analyzing Stress Defense Gene Using Microarray

T3 homozygous cell lines in which the amount of contained spermidine or spermine has been increased 1.1 to 3.0 times compared with the wild type are selected in the transformants. The seeds from the wild type (WT) and the T3 homozygous cell lines (TSP-16, OSP-2) are seeded in plastic pots containing the potting compost (Metromix 250 supplied from Hyponex Japan). Sufficient water is given to the soil, which is then covered with Saran wrap to perform the cold temperature treatment for 2 days (synchronization). The pots after the cold temperature treatment are transferred to a cultivation room, and acclimation for about one week is performed under a long day condition (22° C., 16 hours' light, 50 μmol m⁻² sec⁻¹ PPFD). After one week, the Saran wrap is removed, and the cultivation is started under the above long day condition. On the 50th day (just before internode elongation) after the start of cultivation, an overground part and a root part are separately sampled. Their fresh weights are measured, and immediately they are frozen in liquid nitrogen and stored at −80° C. Total RNA is extracted using TRIZOL reagent (supplied from GIBCO-BRL) in accordance with its protocol. Furthermore, the total RNA is purified using RNeasy column (supplied from Qiagen) in accordance with its protocol. Probes are prepared from 40 μg of 3 kinds of total RNA (WT, TSP-16, OSP-2). The probes are prepared using LabelStar Array labeling kit (supplied from Qiagen) by Cyanine 3-dUTP and Cyanine 5-dUTP in accordance with its protocol. cDNA array chips (donated by Professor Takayuki Kawauchi of Nara Institute of Science and Technology Graduate University) and/or DNA array chips (Arabidopsis supplied from Agilent Technologies) are used for the array analysis. The array chips are prehybridized in prehybridization buffer (4×SSC, 1% BSA, 0.1% SDS) at 37° C. for one hour. The array chips are washed with highly purified Milli Q water. This manipulation is repeated twice. Contained water is removed using a plate centrifuge (1500 rpm, 5 minutes). The array chips are dried in an incubator set at 65° C. for one or more hours. Hybridization is performed using the probe produced by LabelStar Array labeling kit. The array chips are hybridized with hybridization buffer (4×SSC, 10× Denhart solution, 1% BSA, 0.2% SDS, 1 μg/μL of poly A, 0.03 μg/μL of yeast tRNA) containing 35 μL of the probe at 60 to 65° C. for 17 hours. After the hybridization, the array chips are washed with washing solutions starting from 1×SSC and 0.2% SDS at 65° C. to finally 0.2×SSC at room temperature. Scanning and data analysis are performed using Scan Array5000 and QuantArray software (supplied from GSI Lumonics) or ScanArray4000XL (supplied from Packard Biochip Technologies). A fluorescence value of a negative control is used for background, and the background is subtracted from a fluorescence value of each spot. Either a median normalization method or a global normalization method is used for the normalization. In order to increase reliability of the microarray analysis, the analysis is repeated several times for each array chip.

### EXAMPLE 1

### Cloning Spermidine Synthase Gene Derived from Plant

A spermidine synthase gene derived from Cucurbita ficifolia Bouch was acquired in accordance with the description in Example 2 in WO02/23974 (FSPD1, SEQ ID NOS:1 and 2). A spermidine synthase gene (OSPD2, SEQ ID NOS:3 and 4) derived from the rice plant was acquired by the method shown below. In accordance with the description in Example 2 in WO02/23974, the spermidine synthase gene (FSPD1, SEQ ID NOS:1 and 2) derived from Cucurbita ficifolia Bouch, an S-adenosylmethionine decarboxylase gene (FSAM24, SEQ ID NOS:5 and 6) and an arginine decarboxylase gene (FADC76, SEQ ID NOS:7 and 8) were obtained. A spermidine synthase gene (FSPM5, SEQ ID NOS:9 and 10) derived from Arabidopsis thaliana was obtained in accordance with the description in Example 1 in JP 2002-351750 A. The spermidine synthase gene (OSPD2, SEQ ID NOS:3 and 4) derived from the rice plant was acquired by the method shown below.

(1) Preparation of poly (A)+RNA

After removing rice chaff from fully matured seeds of a rice plant cultivar (“Yukihikari”), the seeds were immersed in 70% ethanol for 5 minutes, and subsequently sterilized by immersing them in a sterilization solution (5% sodium hypochlorite, 0.02% Triton X-100) for 20 minutes in a beaker similarly sterilized. The sterilized seeds were washed three times with sterilized water in the sterilized beaker. After washing, the seeds were placed on a growth medium (MS inorganic salts, MS vitamins, 30 g/L of sucrose, 8 g/L of Phytagar, pH 5.8), and cultured in a plant incubator (MLR-350HT, supplied from Sanyo) at 26° C. under a light place condition (45 μmol m⁻² s⁻¹, a light phase for 16 hours and a dark phase for 8 hours, hereinafter this light condition is referred to as the light place). On about 10th day, the low temperature treatment was started by lowering the temperature in the incubator to 12° C. at day and night. On 3 days after the start of the treatment, sampling was performed. The samples were stored at −80° C. until use for RNA extraction.

Young seedlings (about 3 g) were immediately frozen in liquid nitrogen, and finely pulverized in a mortar in the presence of liquid nitrogen. Total RNA was extracted using TRIZOL reagent (supplied from GIBCO-BRL) in accordance with its protocol. A total RNA solution was incubated at 65° C. for 5 minutes, and then rapidly cooled on ice. An equivalent amount of 2× binding buffer (10 mM Tris-HCl, 5 mM EDTA2Na, 1 M NaCl, 0.5% SDS, pH 7.5) was added to this total RNA solution. This mixture was overlaid in an oligo dT cellulose column (supplied from Clontech) previously equilibrated with equilibration buffer (10 mM Tris-HCl, 5 mM EDTA2Na, 0.5 M NaCl, 0.5% SDS, pH 7.5). Then, the column was washed with about 10 time amount of the foregoing equilibration buffer, and subsequently, the poly (A)+RNA was eluted with elution buffer (10 mM Tris-HCl, 5 mM EDTA2Na, pH 7.5). The foregoing aqueous solution of 3 M sodium acetate at 1/10 time amount and ethanol at 2.5 time amount were added to the resulting elution, and the mixture was then left stand at −80° C. Thereafter, the centrifugation at 10,000×g was performed, and the resulting precipitate was washed with 70% ethanol and dried under reduced pressure. This dried preparation was dissolved again in 500 μL of TE buffer, and repeatedly purified on the oligo dT cellulose column. The obtained poly (A)+RNA derived from the young seedlings of the rice plant to which the cold temperature treatment had been given was used for making a cDNA library.

(2) Preparation of cDNA Library

The cDNA library was prepared using a Marathon cDNA Amplification Kit (by Clontech) according to protocol. The poly (A) ⁺RNA was used as template, and reverse transcriptase and modified lock-docking oligo(dT) primer with two degenerate nucleotide positions at the 3′ end were used to synthesize cDNA. A Marathon cDNA adapter (the 5′ end phosphorylated to facilitate binding to both ends of the ds cDNA with T4 DNA ligase) was ligated to both ends of the synthesized cDNA. The resulting adapter-linked cDNA was used as a cDNA library.

(3) Design of PCR Primers

The base sequences of spermidine synthase genes already isolated from plants or mammals were compared. Regions with extremely highly conserved homology were selected to synthesize DNA oligomers (sequence primers I•II)

| | |
|---|---|
| SPDS primer I: | |
| (SEQ ID NO. 11) | 5′-GTTTTGGATGGAGTGATTCA-3′ |
| SPDS primer II: | |
| (SEQ ID NO. 12) | 5′-GTGAATCTCAGCGTTGTA-3′ |

(4) Amplification by PCR

The cDNA library obtained in (2) was used as template, and the sequence primers designed in (3) were used for PCR. The PCR steps involved 5 cycles of 30 seconds at 94° C., 1 minute at 45° C., and 2 minutes at 72° C., followed by 30 cycles of 30 seconds at 94° C., 1 minute at 55° C., and 2 minutes at 72° C.

(5) Agarose Gel Electrophoresis

The PCR amplified products were separated by electrophoresis with 1.5% agarose, and the electrophoresed gel was stained with ethidium bromide to detect amplified bands on a UV transilluminator.

(6) Verification and Recovery of PCR Amplified Products

The detected amplified bands were verified and were cut out of the agarose gel with a razor. The pieces of gel were transferred to 1.5 mL microtubes, and the DNA fragments were isolated and purified from the gel using a QIAEX II Gel Extraction Kit (by QIAGEN). The recovered DNA fragments were subcloned to the pGEMT cloning vector (by Promega), transformed with E. coli, and then used to prepare plasmid DNA in the usual manner.

(7) Sequencing

The sequencing of the sequences inserted into the plasmids were determined by the dideoxy method (Messing, Methods in Enzymol., 101, 20-78 (1983)).

(8) Detection of Homology

A homology search of the base sequences of these genes against a database of known gene base sequences revealed that the obtained genes had 70 to 100% homology with known plant-derived spermidine synthase (SPDS) genes

(9) Isolation of Full Length Gene

The full length gene was isolated by 5′-RACE (rapid amplification of cDNA ends) using Marathon cDNA Amplification Kit (supplied from Clontech) and the method of integrating 3′-RACE (Chenchik et al., 1995). 5′-RACE was performed by PCR with the cDNA library as a template using AP1 primer (5′-CCATCCTAATACGACTCACTATAGGGC-3′) and a primer (5′-TCCCTCGCGTAGCTGTCGGGTTTGA-3′) specific for the gene. The PCR was performed with 35 cycles of 94° C. for 30 seconds, 60° C. for 45 seconds and 72° C. for 2 minutes and then one cycle of 94° C. for 30 seconds, 60° C. for 45 seconds and 72° C. for 7 minutes. 3′-RACE was performed by PCR with the cDNA library as a template using AP1 primer (5′-CCATCCTAATACGACTCACTATAGGGC-3′) and a primer (5′-ACACAACGCCTCCTGGTCGAAGAGC-3′) specific for the gene. The PCR was performed with 35 cycles of 94° C. for 30 seconds, 60° C. for 45 seconds and 72° C. for 2 minutes and then one cycle of 94° C. for 30 seconds, 60° C. for 45 seconds and 72° C. for 7 minutes. Gene fragments obtained in 5′-RACE and 3′RACE were subcloned into pGEM-T cloning vector (supplied from Promega), respectively. Furthermore, all base sequences were determined in accordance with the foregoing method, and analyzed by DINASIS-Mac version 3.6 software package (supplied from Hitachi Software Engineering).

The full length spermidine synthase gene derived from Cucurbita ficifolia Bouch was designated as FSPD1 (SEQ ID NOS:1 and 2), the full length spermidine synthase gene derived from the rice plant was designated as OSPD2 (SEQ ID NOS:3 and 4), the full length S-adenosylmethionine decarboxylase gene was designated as FSAM24 (SEQ ID NOS:5 and 6), the full length arginine decarboxylase gene was designated as FADC76 (SEQ ID NOS:7 and 8), and the full length spermidine synthase gene derived from Arabidopsis thaliana was designated as FSPM5 (SEQ ID NOS:9 and 10).

The obtained FSPD1 and OSPD2 were compared with known spermidine synthase genes derived from the plants at amino acid level, and consequently, FSPD1 was observed to have about 80% homology to the spermidine synthase gene derived from the other plant. OSPD2 was observed to have 100% homology to OsSPDS2 (Journal of Plant Physiology, 161, 883-886, 2004) which was the spermidine synthase gene derived from the rice plant. FSAM24 was compared with known S-adenosylmethionine decarboxylase genes (SAMDC genes) derived from the plants at amino acid level, and consequently FSAM24 was observed to have 63 to 66% homology. FADC76 was compared with known arginine decarboxylase genes derived from the plants, and consequently, FADC76 was observed to have 71 to 77% homology. FSPM5 was compared with spermidine synthase gene (ACL5:GenBank Accession Number AF184093) derived from Arabidopsis thaliana at amino acid level, complete concordance of the amino acids was observed.

### EXAMPLE 2

### Preparation and Analysis of Transgenic Arabidopsis thaliana

(1) Preparation of Expression Construct

The FSPD1 polyamine synthase gene given in SEQ ID NO.1 was cleaved with XhoI in such a way that the entire reading frame of the base sequence was included, and the fragment was purified by the glass milk method. pGEM-7Zf (Promega) was then cleaved with XhoI, and the FSPD1 fragments were subcloned in the sense and antisense directions. The FSPD1 fragments were again cleaved with the XbaI and KpnI restriction enzymes at the multicloning site of pGEM-7Zf, and were each subcloned to the binary vector pBI101-Hm2 to which the 35S promoter (or horseradish peroxidase C2 promoter [U.S. Pat. No. 3,259,178] which was a stress/disease inducible promoter) had been ligated. The resulting plasmid was designated pBI35S-FSPD1+/−, pBIC2-FSPD1+/−. Transformed E. coli JM109 was designated Escherichia coli JM109/pBI35S-FSPD1+/−, Escherichia coli JM109/pBIC2-FSPD1+/−.

The OSPD2 polyamine synthase gene given in SEQ ID NO.3 was cleaved with XhoI in such a way that the entire reading frame of the base sequence was included, and the fragment was purified by the glass milk method. pGEM-7Zf (Promega) was then cleaved with XhoI, and the FSPD1 fragments were subcloned in the sense and antisense directions. The FSPD1 fragments were again cleaved with the XbaI and KpnI restriction enzymes at the multicloning site of pGEM-7Zf, and were each subcloned to the binary vector pBI101-Hm2 to which the 35S promoter (or horseradish peroxidase C2 promoter [U.S. Pat. No. 3,259,178] which was a stress/disease inducible promoter) had been ligated. The resulting plasmid was designated pBI35S-OSPD2+/−, pBIC2-OSPD2+/−. Transformed E. coli JM109 was designated Escherichia coli JM109/pBI35S-OSPD2+/−, Escherichia coli JM109/pBIC2-OSPD2+/−.

The FSAM24 polyamine synthase gene given in SEQ ID NO.5 was cleaved with NotI in such a way that the 5′ nontranslation region (uORF sequence) and the entire reading frame of the base sequence were included, and the ends were blunted. The fragments were subcloned in the sense and antisense directions to the binary vector pBI101-Hm2 to which the (blunted) 35S promoter (or horseradish peroxidase C2 promoter [U.S. Pat. No. 3,259,178] which was a stress/disease inducible promoter) had been ligated. The resulting plasmid was designated pBI35S-FSAM24+/−, pBIC2-FSAM24+/−. Transformed E. coli JM109 was designated Escherichia coli JM109/pBI35S-FSAM24+/−, Escherichia coli JM109/pBIC2-FSAM24+/−.

The FADC76 polyamine synthase gene given in SEQ ID NO.7 was cleaved with NotI in such a way that the 5′ nontranslation region (uORF sequence) and the entire reading frame of the base sequence were included, and the ends were blunted. The fragments were subcloned in the sense and antisense directions to the binary vector pBI101-Hm2 to which the (blunted) 35S promoter (or horseradish peroxidase C2 promoter [U.S. Pat. No. 3,259,178] which was a stress/disease inducible promoter) had been ligated. The resulting plasmid was designated pBI35S-FADC76+/−, pBIC2-FADC76+/−. Transformed E. coli JM109 was designated Escherichia coli JM109/pBI35S-FADC76+/−, Escherichia coli JM109/pBIC2-FADC76+/−.

The FSPM5 polyamine synthase gene given in SEQ ID NO.9 was cleaved with XhoI in such a way that the entire reading frame of the base sequence was included. The fragments were subcloned in the sense and antisense directions to the binary vector pBI101-Hm2 to which the (blunted) 35S promoter (or horseradish peroxidase C2 promoter [U.S. Pat. No. 3,259,178] which was a stress/disease inducible promoter) had been ligated. The resulting plasmid was designated pBI35S-FSPM5+/−, pBIC2-FSPM5+/−. Transformed E. coli JM109 was designated Escherichia coli JM109/pBI35S-FSPM5+/−, Escherichia coli JM109/pBIC2-FSPM5+/−.

(2) Introduction of Plasmids to Agrobacterium

The E. coli pBI35S-FSPD1+/−, E. coli pBIC2-FSPD1+/−, E. coli pBI35S-FSAM24+/−, E. coli pBIC2-FSAM24+/−, E. coli pBI35S-FADC76+/−, E. coli pBIC2-FADC76+/−, E. coli pBI35S-OSPD2+/−, E. coli pBIC2-OSPD2+/−, E. coli pBI35S-FSPM5+/− or E. coli pBIC2-FSPM5+/− obtained in (1) and the E. coli strain HB101 with the pRK2013 helper plasmid were cultured for 1 night at 37° C. on LB medium containing 50 mg/L kanamycin, and the Agrobacterium C58 strain was cultured for 2 nights at 37° C. on LB medium containing 50 mg/L kanamycin. Cells were harvested from 1.5 mL of each culture in Eppendorf tubes and then washed with LB medium. The cells were suspended in 1 mL of LB medium, 100 μL each of the three types of cells were mixed to inoculate LB agar medium and cultured at 28° C. to allow the plasmids to be conjugated with the Agrobacterium (tripartite conjugation). After 1 or 2 days, portions were scraped with a platinum loop and smeared on LB agar medium containing 50 mg/L kanamycin, 20 mg/L hygromycin, and 25 mg/L chloramphenicol. After 2 days of culture at 28° C., a variety of single colonies were selected. The resulting transformants were designated C58/pBI35S-FSPD1+/−, C58/pBIC2-FSPD1+/−, C58/pBI35S-FSAM24+/−, C58/pBIC2-FSAM24+/−, C58/pBI35S-FADC76+/−, C58/pBIC2-FADC76+/−, C58/pBI35S-OSPD2+/−, C58/pBIC2-OSPD2+/−, C58/pBI35S-FSPM5+/− or C58/pBIC2-FSPM5+/−. Transgenic Arabidopsis thaliana was prepared by reduced pressure infiltration ((3) through (6) below).

(3) Cultivation of Arabidopsis thaliana

Potting compost Metromix (Hyponex Japan) was placed in plastic pots, the surfaces were covered with netting mesh, and 2 to 5 seeds (donated by Professor Takayuki Kawauchi of Nara Institute of Science and Technology Graduate University) of Arabidopsis thaliana (referred to below as the “Columbia strain” or “wild type”) were inoculated through the interstices of the mesh. The pots were placed for 2 days at 4° C. in a low temperature chamber to germinate, and were then transferred for cultivation under 22° C. long-day conditions (16 hour long day/8 hour night). After about 4 to 6 weeks, lateral shoots were induced by top pruning plants in which the main axis flower stalk was extended to between 5 and 10 cm. After about 1 to 2 weeks of top pruning, the plants were infected with Agrobacterium.

(4) Preparation of Agrobacterium Suspension

Two days before infection, the Agrobacterium prepared in (2) above was used to inoculate 10 mL LB medium containing antibiotics (50 μg/mL kanamycin, 20 μg/mL hygromycin) for 24 hours of shaking culture at 28° C. Portions of the culture were transferred to 1000 mL LB medium containing antibiotics (50 μg/mL kanamycin, 20 μg/mL hygromycin) for about another 24 hours of shaking culture at 28° C. (to an OD₆₀₀ of between 1.2 and 1.5). Cells were harvested from the culture at ambient temperature and were resuspended in suspension medium for infiltration (0.5×MS salt, 0.5× Gamborg B5 vitamin, 1% sucrose, 0.5 g/L MES, 0.44 μM benzylaminopurine, 0.02% Silwet-77) to an OD₆₀₀ of between 0.8 and 1.

(5) Agrobacterium Infection

The potting soil in the pots of Arabidopsis thaliana prepared in (3) above was watered to prevent the potting soil from absorbing the Agrobacterium suspension prepared in (4) above. Approximately 200 to 300 mL of the Agrobacterium suspension was placed in 1000 mL beakers, and the potted Arabidopsis thaliana was turned upside down to dip the plants in the suspension. The beakers in which the pots had been placed were put into a dessicator, which was suctioned with a vacuum pump to about −0.053 MPa (400 mmHg), and the plants were then allowed to stand for about 10 minutes. The negative pressure was gradually released, the plants were then taken out of the Agrobacterium suspension, the excess Agrobacterium suspension was wiped off with a Kimtowel, and the pots were placed on their sides in deep-bottomed trays. A small amount of water was introduced, and the plants were covered with saran wrap. The plants were allowed to stand in this manner for about 1 day. The saran wrap was then removed, and the pots were placed upright and irrigation was stopped for about 1 week. The potting compost was then gradually watered, and seeds were harvested from matured pods for about 3 to 5 weeks. The harvested seeds were strained through a tea strainer to eliminate debris and husks, and the seeds were placed in a dessicator and thoroughly dried.

(6) Obtaining Transformed Plants

100 μL (about 2000) seeds obtained in (5) above were transferred to 1.5 mL Eppendorf tubes and soaked for 2 minutes in 70% ethanol and 15 minutes in 5% sodium hypochlorite solution, and the seeds were finally washed five times with sterile water to disinfect the seeds. The disinfected seeds were transferred to 15 mL falcon tubes, about 9 mL of 0.1% aseptic agar solution was added, and the contents were vigorously mixed. A 0.1% agar mixture of seeds was evenly spread on selection medium (1×MS salt, 1× Gamborg B5 vitamin, 1% sucrose, 0.5 g/L MES, 0.8% agar, 100 mg/L carbenicillin, 50 mg/L kanamycin, 40 mg/L hygromycin, 8 g/L Phytagar, pH 5.7) like plating the phages. The plates were dried for about 30 minutes in a clean bench, a 4° C. low temperature treatment was performed for 2 days, the plates were transferred to a 22° C. growth chamber, and transformants with antibiotic resistance were selected. Plants with about 3 to 5 true leaves were again transferred to fresh selection medium and cultivated until 4 to 6 true leaves had grown. Transformants with antibiotic resistance (T1) were planted in pots filled with compost and acclimated under humid conditions for about 5 to 7 days. After acclimation, the plants were cultivated at 23° C. under long day conditions (16 hour long days/8 hour nights). The resulting transformed plants (T1) and plants T2 grown from seeds (T2) obtained from the transformed plants were analyzed for genes introduced by PCR or Southern hybridization and their levels of expression by Northern hybridization were analyzed, and transformants which are confirmed that the target spermidine synthase genes had been incorporated in a stable manner and expressed was selected. Seeds T3 were also harvested from the plants T2, and antibiotic resistance tests (segregation analysis) were conducted to obtain homozygotes (T2) based on the proportion in which transformants appeared. Seeds T2 and seeds T3 obtained from the homozygotes (T3 homozygous cell line) were used in the following tests.

(7) Northern Blotting Analysis

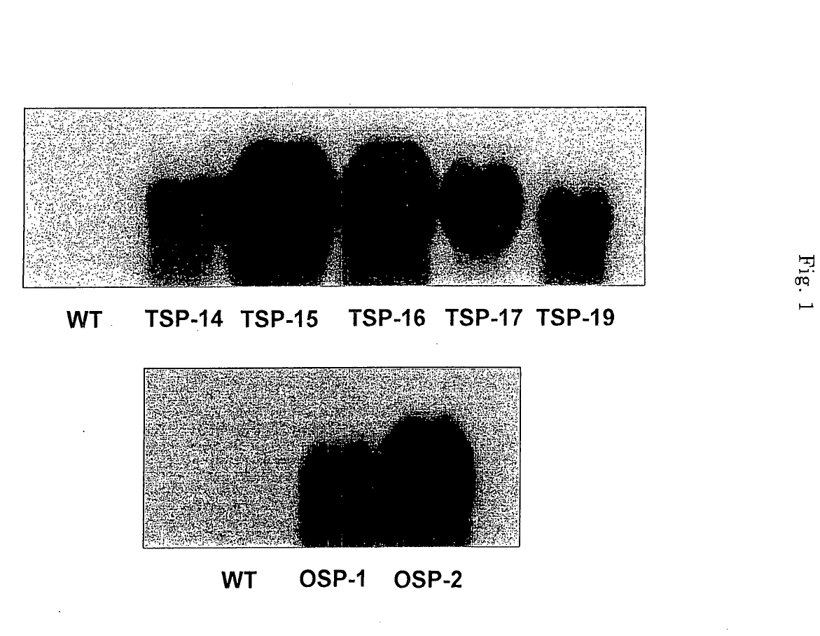

In order to confirm expression levels of FSPD1 and OSPD2 in T2 transformants obtained in (6), Northern blotting was performed. Total RNA was extracted from untransformed wild type (WT) and T2 transformant (FSPD1 introduced cell lines: TSP-14, 15, 16, 17, 19; OSPD2 introduced cell lines: OSP-1, 2) rosette leaves. The RNA extraction was performed according to an ordinary method. 10 μg of the resulting total RNA was electrophoresed on 1.5% formaldehyde agarose gel and blotted over night on HyBond N nylon membranes. The RNA was fixed with a UV crosslinker and then pre-hybridized for 2 hours at 42° C. in pre-hybridization buffer (50% formamide, 5×SSPE, 5× Denhardt's, 0.1% SDS, 80 μg/mL salmon sperm DNA, pH 7.0). Probes were prepared with the use of ³²P-dCTP and a random label kit (by Amersham) from the cDNA of the rice SPDS gene fracment and Cucurbita ficifolia Bouche SPDS gene fragment obtained in (6) of Example 1. The probe was added to the pre-hybridization mixture for hybridization over night at 42° C. After the hybridization, the membranes were washed twice for 30 minutes at 55° C., beginning with a washing solution containing 2×SSC and 0.1% SDS, for 30 minutes at 50° C. with a washing solution containing 0.5×SSC and 0.1% SDS, and ending with a washing solution containing 0.1×SSC and 0.1% SDS. Autoradiographs of the membranes were taken using X-ray film (Kodak). Part of the results of Northern blotting are given in FIG. 1. The results in FIG. 1 show that no expression of the exogenous Cucurbita ficifolia Bouche SPDS gene (FSPD1) or rice SPDS gene (OSPD2) was detected in the wild type (WT), but that signals were detected in all the cell lines at high levels, and expression of FSPD1 and OSPD2 was confirmed.

(8) Polyamine Analysis

Cell lines were selected from the results of PCR (or Southern analysis), Northern analysis or Western analysis. Polyamine analysis was performed for the cell lines in which the polyamine metabolism-relating enzyme gene had been absolutely introduced and the gene was stably expressed. The cell lines, TSP-14, TSP-15, TSP-16, TSP-17, TSP-19 and TSP-101, in which FSPD1 had been introduced in a sense direction, the cell lines, TSA-1 and TSA-4 in which FSAM24 had been introduced in the sense direction, the cell lines, TAD-3 and TAD-5, in which FADC76 had been introduced in the sense direction, and the cell lines, TSM-3 and TSM-7, in which FSPM5 had been introduced in the sense direction were selected. The cell lines, OSP-1, OSP-2, OSP-5 and OSP-7, in which OSPD2 had been introduced in the sense direction were selected. About 0.1 to 0.5 g of rosette leaves were sampled from wild type (WT) and transformants (TSP, OSP), and stored frozen. Diluted internal standards (1,6-hexanediamine, internal standard amount=7.5 or 12 nmol) as well as 5% perchloric acid aqueous solution (5 to 20 mL per 1.0 g live weight of sample) were added to the samples, which were thoroughly milled and extracted at ambient temperature in an omnimixer. The milled solution was centrifuged at 35,000×g at 4° C. for 20 minutes, a supernatant solution was collected, and the solution was made a free polyamine solution. The free polyamine solution (400 μL), 200 μL of saturated aqueous solution of sodium carbonate and 200 μL of dansyl chloride/acetone solution (10 mg/mL) were added into a microtube with a screw cap, and gently mixed. The cap of the tube was tightly sealed followed by being covered with aluminium foil, and the mixture was heated in a water bath at 60° C. for one hour to perform dansylation. After cooling the tube, 200 μL of an aqueous solution of proline (100 mg/mL) was added and mixed. The tube was covered with aluminium foil and heated again in the water bath for 30 minutes. After cooling, acetone was removed by blowing nitrogen gas, then 600 μL of toluene was added and mixed vigorously. The tube was left stand to separate two phases, and subsequently, 300 to 400 μL of an upper layer toluene layer was dispensed in a microtube. Toluene was completely removed by blowing the nitrogen gas to the dispensed toluene. The dansylated free polyamine was dissolved by adding 200 μL of methanol to the tube. Putrescine, spermidine and free polyamine of spermine were quantified by an inner standard method using high performance liquid chromatography connecting a fluorescence detector (excitation wavelength: 365 nm, luminescence wavelength: 510 nm). The HPLC column was a μBondapak C18 (027324 by Waters, 3.9×300 mm, 10 μm particle diameter). The polyamine content of the samples was calculated by determining the peak area of the internal standard and each polyamine based on the HPLC chart of the standard solutions and samples. The results are given in Table 2.

TABLE 2

| | Free polyamine content (nmolg⁻¹fw) | | | |
|---|---|---|---|---|
| Cell | | | | Total |
| line | Putrescine | Spermidine | Spermine | polyamines |
| Wild | 5.41 ± 3.74 | 108.99 ± 12.63 | 11.95 ± 2.92 | 126.35 ± 16.04 |
| type: | | | | |
| WT | | | | |
| TSP-14 | 6.06 ± 3.04 | 149.96 ± 11.64 | 23.68 ± 2.06 | 179.70 ± 20.82 |
| TSP-15 | 8.33 ± 2.05 | 175.76 ± 16.30 | 21.53 ± 1.29 | 205.62 ± 20.82 |
| TSP-16 | 10.66 ± 3.98 | 182.94 ± 23.73 | 21.36 ± 6.48 | 214.96 ± 29.41 |
| TSP-17 | 12.40 ± 3.89 | 177.45 ± 12.70 | 13.33 ± 1.07 | 203.18 ± 16.77 |
| TSP-19 | 7.82 ± 3.55 | 169.13 ± 36.97 | 21.59 ± 3.10 | 198.54 ± 41.49 |
| TSP- | 6.89 ± 5.15 | 298.99 ± 20.11 | 29.89 ± 3.09 | 335.77 ± 24.51 |
| 101 | | | | |
| TSA-1 | 7.02 ± 3.10 | 165.55 ± 11.54 | 19.11 ± 2.00 | 191.68 ± 14.66 |
| TSA-4 | 8.34 ± 3.65 | 162.34 ± 24.01 | 19.04 ± 7.01 | 189.72 ± 29.89 |
| TAD-3 | 10.99 ± 3.82 | 159.39 ± 12.45 | 18.98 ± 1.51 | 189.36 ± 15.89 |
| TAD-5 | 11.98 ± 3.72 | 158.99 ± 12.93 | 18.54 ± 2.90 | 189.51 ± 16.54 |
| TSM-3 | 8.20 ± 3.76 | 134.55 ± 15.67 | 30.21 ± 8.07 | 172.96 ± 23.43 |
| TSM-7 | 9.23 ± 2.98 | 152.09 ± 21.00 | 33.76 ± 9.11 | 195.08 ± 26.20 |
| OSP-1 | 5.02 ± 3.21 | 175.45 ± 10.65 | 20.14 ± 2.00 | 200.61 ± 14.56 |
| OSP-2 | 7.74 ± 3.56 | 199.22 ± 20.19 | 19.41 ± 7.51 | 226.37 ± 29.87 |
| OSP-5 | 10.55 ± 3.22 | 179.21 ± 11.54 | 18.97 ± 2.51 | 208.73 ± 14.88 |
| OSP-7 | 11.88 ± 2.92 | 148.99 ± 15.93 | 19.33 ± 3.90 | 180.20 ± 17.45 |

As is shown in Table 2, it has been demonstrated that the amounts of putrescine, spermidine and spermine contained in the cell lines in which FSPD1, FSAM24, FADC76, FSPM5 or OSPD2 which was the spermidine synthase gene had been introduced in the sense direction were significantly increased compared with those in the wild type (WT) and that the amounts of total polyamine contained were also significantly increased compared with that in the wild type (WT). In particular, the amounts of contained spermidine and spermine were remarkably increased. It has been shown that the amounts of contained spermidine and spermine were increased in the range of 1.1 to 3.0 times compared with those in the wild type (non-transformant) by introducing FSPD1, FSAM24, FADC76, FSPM5 or OSPD2 into the plant. Adverse effects such as growth inhibition and fertility reduction were not observed in the transformants (cell lines) in which the amounts of contained spermidine and spermine had been increased in the range of 1.1 to 3.0 times compared with those in the wild type (non-transformant).

### EXAMPLE 3

### Microarray Analysis of Transgenic Arabidopsis

T3 homozygous cell lines in which the amounts of contained spermidine and spermine had been increased in the range of 1.1 to 3.0 times compared with those in the wild type were selected in the T3 transformants. The seeds from the wild type (WT) and the T3 homozygous cell lines (TSP-16, OSP-2) were seeded in plastic pots containing the potting compost (Metromix 250 supplied from Hyponex Japan). Sufficient water was given to the soil, and the pots were covered with Saran wrap, to which the cold temperature treatment (synchronization) was given for 2 days. After the cold temperature treatment, the pots were transferred to the cultivation room, and the acclimation for about one week was performed under the long day condition (22° C., lighten for 16 hours, 50 μmol m⁻² sec⁻¹ PPFD). After one week, the Saran wrap was removed, and the cultivation was started under the above long day condition. On the 50th day (just before internode elongation) after the start of the cultivation, an overground part and a root part were separately sampled. Their fresh weights were measured, and immediately they were frozen in liquid nitrogen and stored at −80° C. Total RNA was extracted using TRIZOL reagent (supplied from GIBCO-BRL) in accordance with its protocol. Furthermore, the total RNA was purified using RNeasy column (supplied from Qiagen) in accordance with its protocol. Probes were prepared from 40 μg of 3 kinds of total RNA (WT, TSP-16, OSP-2). The probes were prepared using LabelStar Array labeling kit (supplied from Qiagen) by Cyanine 3-dUTP and Cyanine 5-dUTP in accordance with its protocol. cDNA array chips (donated by Professor Takayuki Kawauchi of Nara Institute of Science and Technology Graduate University) and/or DNA array chips (Arabidopsis supplied from Agilent Technologies) were used for the array analysis. The array chips were prehybridized in prehybridization buffer (4×SSC, 1% BSA, 0.1% SDS) at 37° C. for one hour. The array chips were washed with highly purified Milli Q water. This manipulation was repeated twice. Contained water was removed using the plate centrifuge (1500 rpm, 5 minutes). The array chips were dried in the incubator set at 65° C. for one or more hours. Hybridization was performed using the probe produced by the LabelStar Array labeling kit. The array chips were hybridized with hybridization buffer (4×SSC, 10× Denhart solution, 1% BSA, 0.2% SDS, 1 μg/μL of poly A, 0.03 μg/μL of yeast tRNA) containing 35 μL of the probe at 60 to 65° C. for 17 hours. After the hybridization, the array chips were washed with washing solutions starting from 1×SSC and 0.2% SDS at 65° C. to finally 0.2×SSC at room temperature. Scanning and data analysis were performed using Scan Array5000 and QuantArray software (supplied from GSI Lumonics) or ScanArray4000XL (supplied from Packard Biochip Technologies). The fluorescence value of the negative control was used for the background, and the background was subtracted from the fluorescence value of each spot. Either the median normalization method or the global normalization method was used for the normalization. In order to increase reliability of the microarray analysis, the analysis was repeated several times for each array chip. The stress defense genes whose expression amounts had been increased (Ratio of expression amount is in the range of 1.5 to 5.0 times) in the transformants (TSP-16, OSP-2) compared with those in the wild type (WT) were shown in Table 3.

TABLE 3

| | | Ratio of | |
|---|---|---|---|
| | | expression | |
| | | amount | |
| Gene | | (TSP or | Accession |
| number | Stress defense gene | OSP/WT) | Number |
| 1 | transcription factor/CBF1, DREB1B | 1.8 ± 0.3 | AT4G25490 |
| 2 | cold regulated protein/LEA protein | 4.2 ± 1.0 | AT2G03740 |
| 3 | cold regulated protein/cor15 | 1.6 ± 0.3 | AT2G42530 |
| 4 | pathogen related PR-1 protein/PR-1 | 3.3 ± 1.3 | AT2G14610 |
| 5 | early response dehydration protein/ | 2.0 ± 0.5 | D30719 |
| | ERD15 | | |
| 6 | salt stress induced tonoplast intrinsic | 2.3 ± 0.6 | AF004393 |
| | protein/aquaporin | | |
| 7 | water channel protein/aquaporin | 2.2 ± 0.3 | AAC79629 |
| 8 | dehydration induced protein/RD22, | 1.5 ± 0.4 | AT5G25610 |
| | rd22 | | |
| 9 | stress responsive protein | 3.9 ± 1.1 | CAB52439 |
| 10 | drought induced protein | 2.8 ± 0.7 | AT1G72290 |
| 11 | low temperature and salt responsive | 2.5 ± 0.6 | CAB79783 |
| | protein | | |
| 12 | stress responsive protein | 2.1 ± 0.2 | CAB52439 |
| 13 | zinc finger protein | 2.1 ± 0.4 | AT5G43170 |
| 14 | disease resistance protein | 2.6 ± 0.5 | BAB08633 |
| 15 | disease resistance protein | 2.3 ± 0.2 | AT3G05660 |
| 16 | disease resistance protein | 2.3 ± 0.3 | BAB09430 |
| 17 | disease resistance protein | 2.2 ± 0.3 | AT5G18350 |
| 18 | disease resistance protein | 2.2 ± 0.2 | AT4G11210 |
| 19 | Peroxidase | 2.3 ± 0.5 | AT4G33420 |
| 20 | senescence associated protein sen1 | 2.2 ± 0.5 | AT4G35770 |

From the results in Table 3, it has been shown that the expression levels in the stress defense gene group (Gene Numbers 1 to 20) are increased 1.5 to 5.0 times compared with those in the wild type (WT) which is the non-transformant by transforming the plant with the spermidine synthase gene (FSPD1) derived from Cucurbita ficifolia Bouch or the spermidine synthase gene (OSPD2) derived from the rice plant.

It has been reported that a CBF1/DREB1B transcription factor of Gene Number 1 is one of the transcription factors induced by stress and that tolerance against various environmental stress such as drought, salt, freeze and cold temperature is enhanced by introducing it into the plants (The Plant Cell, 10, 1391-1406, 1998, Nature Biotechnology, 17, 287-291, 1999, Plant Physiology, 124, 1854-1865, 2000, Plant Physiology, 130, 639-648, 2002, Plant Physiology, 130, 618-626, 2002).

It has been known that an old regulated protein/LEA protein of Gene Number 2 is a late embryogenesis abundant (LEA) protein and induced by stress, and it has been reported that the tolerance against drought stress and salt stress is enhanced by introducing HVI which is the LEA protein gene into the rice plant (Plant Physiology, 110, 249-257, 1996).

It has been reported that a cold regulated protein/cor15 of Gene Number 3 is the gene induced by cold temperature stress and that it is deeply involved in the freeze stress tolerance (Pro. Natl. Acad. Sci. USA, 93, 13404-13409, 1996, Pro. Natl. Acad. Sci. USA, 95, 14570-14575, 1998).

It has been reported that a pathogen related PR-1 protein of Gene Number 4 is the protein induced by pathogen infection and that the tolerance against heavy metal stress and pathogen infection stress is enhanced by introducing the CABPR1 gene which is one of PR-1 (pathogenesis-related protein 1) into tobacco (Plant Cell Rep., Feb. 18, 2005).

It has been reported that an early response dehydration protein/ERD15 of Gene Number 5 is the gene induced by drought stress and is deeply involved in the drought stress tolerance (Plant Physiology, 106, 1707, 1994).

It has been reported that a salt stress induced tonoplast intrinsic protein/aquaporin of Gene Number 6 and a water channel protein/aquaporin are the water channel proteins induced by stress and are deeply involved in the tolerance against osmotic stress and low temperature stress (Mol. Cells., 9(1), 84-90, 1999, Foods Food Ingredients J. Jpn., 176, 40-45).

It has been reported that a dehydration induced protein/RD22, rd22 of Gene Number 8 is the protein induced by drought stress and is deeply involved in the drought stress tolerance (Plant Cell., 15(1), 63-78, 2003).

The proteins of Gene Numbers 9, 10, 11, 12 and 13 are the proteins induced by stress, and it has been suggested that they are involved in stress tolerance, but their functions are not elucidated sufficiently.

The proteins of Gene Numbers 14, 15, 16, 17 and 18 are the proteins induced by disease stress, and it has been suggested that they are involved in the disease stress tolerance, but their functions are not elucidated sufficiently.

It has been known that peroxidase of Gene Number 19 is one (EC 1.11.1.7) of cell wall enzymes and induced by disease stress, and it has been reported that tolerance against oxidative stressoxidative stress and pest stress is enhanced by introducing it into the plant (Plant Physiology, 132, 1177-1185, 2003, J. Econ. Entomol., 95(1), 81-88, 2002).

It has been reported that a senescence associated protein sen1 of Gene Number 20 is the protein induced by aging stress, salt stress, osmotic stress and low temperature stress and is deeply involved in tolerance against aging stress, salt stress, osmotic stress and low temperature stress (Plant Physiology, 130, 2129-2141, 2002).

From the above results, it has been demonstrated that the expression amount of the stress defense gene involved in the stress tolerance is increased (induction or increase) compared with that in the wild type by transforming the plant with the polyamine synthase gene, particularly the spermidine synthase (SPDS) gene.

Next, the expression amounts of the stress defense genes under the stress condition were compared between the transformants and the wild type. T3 homozygous cell lines in which the amounts of contained spermidine and spermine had been increased in the range of 1.1 to 3.0 times compared with that in the wild type were selected in the T3 transformants. The seeds from the wild type (WT) and the T3 homozygous cell lines (TSP-16, OSP-2) were seeded in plastic pots containing the potting compost (Metromix 250 supplied from Hyponex Japan). Sufficient water was given to the soil, and the pots were covered with Saran wrap, to which the cold temperature treatment (synchronization) was given for 2 days. After the cold temperature treatment, the pots were transferred to the cultivation room, and the acclimation for about one week was performed under the long day condition (22° C., lighten for 16 hours, 50 μmol m⁻² sec⁻¹ PPFD). After one week, the Saran wrap was removed, and the cultivation was started under the above long day condition. On the 48th day (just before internode elongation) after the start of the cultivation, the pots were transferred under the low temperature stress condition (5/5° C.: day/night, lighten for 16 hours, 240 μmol m⁻² sec⁻¹ PPFD) to perform the stress treatment for 2 days. After the treatment, an overground part and a root part were separately sampled. Their fresh weights were measured, and immediately they were frozen in liquid nitrogen and stored at −80° C. Total RNA was extracted using TRIZOL reagent (supplied from GIBCO-BRL) in accordance with its protocol. Furthermore, the total RNA was purified using RNeasy column (supplied from Qiagen) in accordance with its protocol. Probes were prepared from 40 μg of 3 kinds of total RNA (WT, TSP-16, OSP-2). The probes were prepared using LabelStar Array labeling kit (supplied from Qiagen) by Cyanine 3-dUTP and Cyanine 5-dUTP in accordance with its protocol. cDNA array chips (donated by Professor Takayuki Kawauchi of Nara Institute of Science and Technology Graduate University) and/or DNA array chips (Arabidopsis supplied from Agilent Technologies) were used for the array analysis. The array chips were prehybridized in prehybridization buffer (4×SSC, 1% BSA, 0.1% SDS) at 37° C. for one hour. The array chips were washed with highly purified Milli Q water. This manipulation was repeated twice. Contained water was removed using the plate centrifuge (1500 rpm, 5 minutes). The array chips were dried in the incubator set at 65° C. for one or more hours. Hybridization was performed using the probe produced by the LabelStar Array labeling kit. The array chips were hybridized with hybridization buffer (4×SSC, 10× Denhart solution, 1% BSA, 0.2% SDS, 1 μg/μL of poly A, 0.03 μg/μL of yeast tRNA) containing 35 μL of the probe at 60 to 65° C. for 17 hours. After the hybridization, the array chips were washed with washing solutions starting from 1×SSC and 0.2% SDS at 65° C. to finally 0.2×SSC at room temperature. Scanning and data analysis were performed using Scan Array5000 and QuantArray software (supplied from GSI Lumonics) or ScanArray4000XL (supplied from Packard Biochip Technologies). The fluorescence value of the negative control was used for the background, and the background was subtracted from the fluorescence value of each spot. Either the median normalization method or the global normalization method was used for the normalization. In order to increase reliability of the microarray analysis, the analysis was repeated several times for each array chip. The stress defense genes whose expression amounts had been increased (Ratio of expression amount is in the range of 2.0 to 18.0 times) in the transformants (TSP-16, OSP-2) compared with those in the wild type (WT) were shown in Table 4.

TABLE 4

| | | Ratio of | |
|---|---|---|---|
| | | expression | |
| | | amount | |
| Gene | | (TSP or | Accession |
| number | Stress defense gene | OSP/WT) | number |
| 21 | senescence associated protein | 17.9 ± 2.1 | BAB33421 |
| 22 | nematode resistance protein/ | 9.5 ± 1.7 | NP181529 |
| | Hs1pro-1 | | |
| 23 | WRKY transcription factor | 5.6 ± 1.3 | AT4G23810 |
| 24 | RMA1 RING zinc finger protein | 4.1 ± 0.8 | BAA28598 |
| 25 | transcriptional activator CBF1/ | 3.5 ± 0.5 | AT1G12630 |
| | CBF1 | | |
| 26 | zinc finger protein | 3.0 ± 0.6 | AT3G07650 |
| 27 | transcription factor/DREB1A | 2.8 ± 0.4 | AT1G63030 |
| 28 | transcription factor/DREB1A | 2.6 ± 0.4 | AT1G63040 |
| 29 | stress induced protein sti1 | 2.6 ± 0.3 | T48150 |
| 30 | early response dehydration | 2.6 ± 0.4 | T02438 |
| | protein/ERD15 | | |
| 31 | B-box zinc finger protein | 2.6 ± 0.4 | AT1G68520 |
| 32 | transcription factor/DREB2B | 2.5 ± 0.3 | AT3G11020 |
| 33 | heat shock protein DnaJ | 2.5 ± 0.2 | AT4G36040 |
| | homolog | | |
| 34 | pathogenesis related protein | 2.5 ± 0.3 | T04989 |
| 35 | myb protein | 2.4 ± 0.3 | AT4G372760 |
| 36 | jasmonic acid regulatory protein | 2.4 ± 0.4 | AAF35416 |
| 37 | early response dehydration | 2.3 ± 0.5 | D30719.1 |
| | protein/ERD15 | | |
| 38 | low temperature induced | 2.3 ± 0.4 | AT5G52310 |
| | protein 78/LTI78, rd29A, | | |
| | COR78 | | |
| 39 | salt tolerance zinc finger protein | 2.2 ± 0.3 | CAA64820 |
| 40 | CCCH type zinc finger protein | 2.2 ± 0.3 | AT2G25900 |
| 41 | Cytochrome P450 | 2.2 ± 0.2 | AT5G45340 |
| 42 | transcription activator | 2.1 ± 0.4 | AT1G12610 |
| | CBF1/CBF1 | | |
| 43 | DREB like AP2 domain | 2.1 ± 0.2 | AT2G38340 |
| | transcription factor/DREB2E | | |
| 44 | Peroxidase | 2.0 ± 0.3 | AT5G64120 |
| 45 | AP2 domain protein | 2.0 ± 0.3 | AT1G78080 |
| 46 | senescence associated protein | 2.0 ± 0.4 | AT4G35770 |
| | sen1 | | |
| 47 | stress responsive protein | 2.0 ± 0.4 | CAB52439 |
| 48 | zinc finger protein | 2.0 ± 0.5 | AT5G43170 |
| 49 | disease resistance protein | 2.0 ± 0.3 | BAB08633 |

From the results in Table 4, it has been shown that the expression levels in the stress defense gene group (gene Numbers 1 to 20) are increased 2.0 to 18.0 times compared with those in the wild type (WT) even under the stress condition by transforming the plant with the polyamine synthase gene, particularly the spermidine synthase gene (FSPD1) derived from Cucurbita ficifolia Bouch or the spermidine synthase gene (OSPD2) derived from the rice plant.

It has been reported that the senescence associated proteins of Gene Numbers 21 and 46 are induced by aging stress, salt stress, osmotic stress and low temperature stress and are deeply involved in the tolerance against aging stress, salt stress, osmotic stress and low temperature stress (Plant Physiology, 130, 2129-2141, 2002).

It has been reported that the nematode resistance protein/Hslpro-1 of Gene Number 22 is deeply involved in nematode stress tolerance (Science, 275, 832-834, 1997).

It has been reported that a WRKY transcription factor of Gene Number 23 is one of transcription factors and is deeply involved in pathogen stress tolerance (EMBO J., 15, 5690-5700, 1996, Plant Mol., Biol., 29, 691-702, 1995, Mol. Plant-Microbe Interact., 16, 295-305, 2003), drought stress tolerance and high temperature stress tolerance (Plant Physiol., 130, 1143-1151, 2002).

It has been reported that the zinc finger protein of gene Number 24, 26, 31, 39, 40 or 48 is one of transcription factors induced by salt stress, osmotic stress and low temperature stress and is deeply involved in the tolerance against salt stress, osmotic stress and low temperature stress (Plant Physiology, 130, 2129-2141, 2002).

It has been reported that a transcriptional activator CBF1, a transcription activator CBF1 and transcription factor DREB of Gene Numbers 25, 42, 27, 28 and 32 are the transcription factors induced by various stresses such as salt stress, osmotic stress and low temperature stress (Plant Physiology, 130, 2129-2141, 2002) and that the tolerance against various environmental stresses such as drought, salt, freeze and low temperature is enhanced by introducing them into the plants (The Plant Cell, 10, 1391-1406, 1998, Nature Biotechnology, 17, 287-291, 1999, Plant Physiology, 124, 1854-1865, 2000, Plant Physiology, 130, 639-648, 2002, Plant Physiology, 130, 618-626, 2002).

The stress induced protein stil and the stress responsive protein of Gene Numbers 29 and 47 are the proteins induced by the stress and it has been suggested that they are involved in the stress tolerance, but their functions are not elucidated sufficiently.

It has been reported that the early response dehydration protein/ERD15 of Gene Numbers 30 and 37 is induced by drought stress and is deeply involved in drought stress tolerance (Plant Physiology, 106, 1707, 1994).

It has been reported that the heat shock protein DnaJ homolog of Gene Number 33 is the gene induced by high temperature stress, drought stress and low temperature stress and is deeply involved in the tolerance against high temperature stress, drought stress and low temperature stress (The Plant Cell, 13, 61-72, 2001).

It has been reported that the pathogenesis related protein of Gene Number 34 is induced by pathogen infection and that the tolerance against heavy metal stress and pathogen infection stress is enhanced by introducing CABPR1 gene which is one of PR-1 (pathogenesis-related protein) into tobacco (Plant Cell Rep., Feb. 18, 2005).

It has been reported that the myb protein of gene Number 35 is one of transcription factors, is the protein induced by ABA (abscisic acid), drought stress and low temperature stress, and is deeply involved in the tolerance against drought stress and low temperature stress (The Plant Cell, 5, 1529-1539, 1993, The Plant Cell, 9, 1859-1868, 1997, Plant Physiology, 130, 2129-2141, 2002).

It has been reported that the jasmonic acid regulatory protein of Gene Number 36 is induced by jasmonic acid and is deeply involved in the tolerance against pathogen stress, pest stress and disease stress because jasmonic acid is involved in pathogen stress, pest stress and disease stress (Trends Plant Sci., 2, 302-307, 1997).

It has been reported that the low temperature induced protein 78/LTI78, rd29A, COR78 of Gene Number 38 is induced by low temperature stress, drought stress and salt stress and is deeply involved in the tolerance against low temperature stress, drought stress and salt stress (Plant Cell, 6,251-264, 1994).

Cytochrome P450 of Gene Number 42 is the protein induced by various stresses, and is suggested to be involved in various stresses.

It has been reported that the AP2 domain transcription factor and the AP2 domain protein of Gene Numbers 43 and 45 are the transcription factors induced by low temperature stress and are deeply involved in the tolerance against low temperature stress (Plant Physiology, 130, 2129-2141, 2002, The Plant Journal, 38, 9820993, 2004).

It has been known that peroxidase of Gene Number 44 is one (EC 1.11.1.7) of cell wall enzymes and induced by the disease stress, and it has been reported that the tolerance against oxidative stressoxidative stress and pest stress is enhanced by introducing it into the plant (Plant Physiology, 132, 1177-1185, 2003, J. Econ. Entomol., 95(1), 81-88, 2002).

Gene Number 49 is the protein induced by the disease stress and has been suggested to be involved in the disease stress tolerance, but its functions are not elucidated sufficiently.

### EXAMPLE 4

### Evaluation of Stress Defense Capacity

(1) Evaluation of Osmotic Stress Tolerance

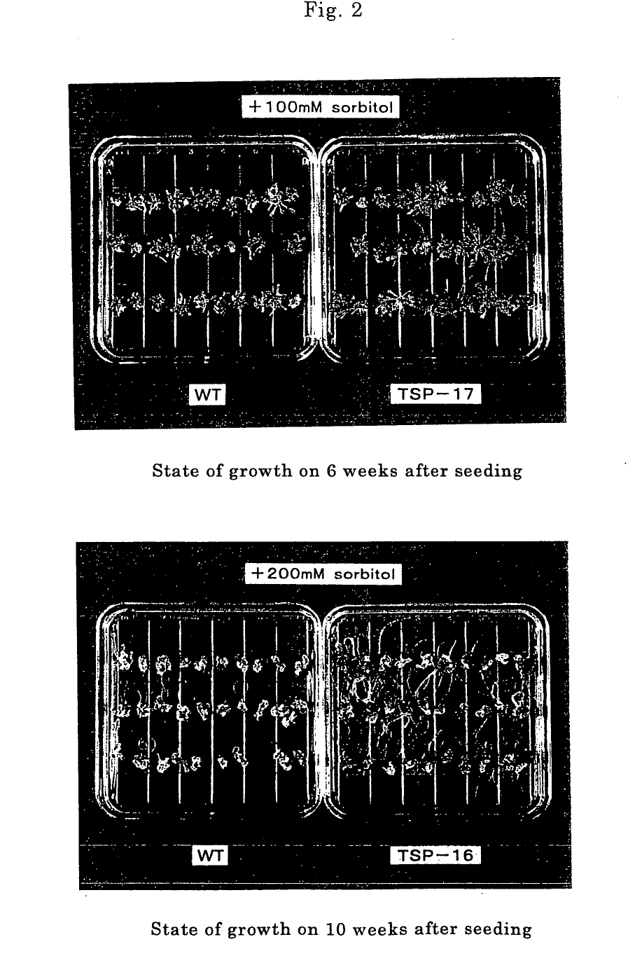

The surfaces of seeds of the transformants (TSP-15, 16, 17) obtained in Example 2 and the wild type (WT: Columbia strain) were sterilized in the same manner as in section (6) of Example 2. Germination growth media containing 100 mM and 200 mM sorbitol (1×MS salt, 10 g/L sucrose, 0.1 g/L myo-inositol, 5% MES, 8 g/L Phytagar, pH 5.7) was inoculated with the sterilized seeds one at a time. Inoculation was followed by about 2 days of low temperature treatment at 4° C., and then by the start of cultivation at 22° C. under conditions involving long days (16 hour long days/8 hour nights). The state of growth after inoculation was monitored, particularly the state of the growth of plants on germination growth media during weeks 6 and 10. The results are given in FIG. 2.

Several days following inoculation, TSP-15, 16, and 17 showed improved germination than the wild type (WT) on growth medium containing 100 mM and 200 mM sorbitol, revealing improved growth. In week 6 after inoculation, TSP-15, 16, and 17 plants on medium containing 100 mM and 200 mM sorbitol were larger than the WT, with significantly less impaired growth. The results for TSP-17 in particular are given in FIG. 2. After week 7 following inoculation, the plants on TSP-15, 16, and 17 containing 200 mM sorbitol in particular exhibited far improved growth, particularly the roots, compared to WT. In week 10 following inoculation, there were significant differences in both the parts above ground and the roots. The results for TSP-16 in particular are given in FIG. 2. Some of the WT were found to have yellowed and died due to impaired growth.

From the above results, it has been demonstrated that the osmotic stress defense effect can be imparted to the plant by introducing the spermidine synthase gene (FSPD1, OSPD2).

(2) Evaluation of Drought Stress Tolerance

The transformants (T3 homozygous cell lines) selected from the transformants (TSP-15, TSP-16) were used. The seeds from the obtained transformants and the wild type (WT: Columbia strain) were seeded in plastic pots containing the potting compost, Metromix (supplied from Hyponex Japan). Inoculation was followed by about 2 days of low temperature treatment at 4° C., and the pots were then placed in plastic vats to start cultivation at 23° C. under conditions involving long days (16 hour long days/8 hour nights). Rosette leaves had fully developed by about week 3 after inoculation. Individuals characterized by uniform growth at the time the rosette leaves had fully developed were selected, water was then fed into the vats to ensure uniform soil moisture, and water was filled to the middle of the plastic pots. After 5 days, a constant soil moisture was confirmed, and drought stress treatment was started (termination of water feed). The state of growth was monitored immediately after water termination.

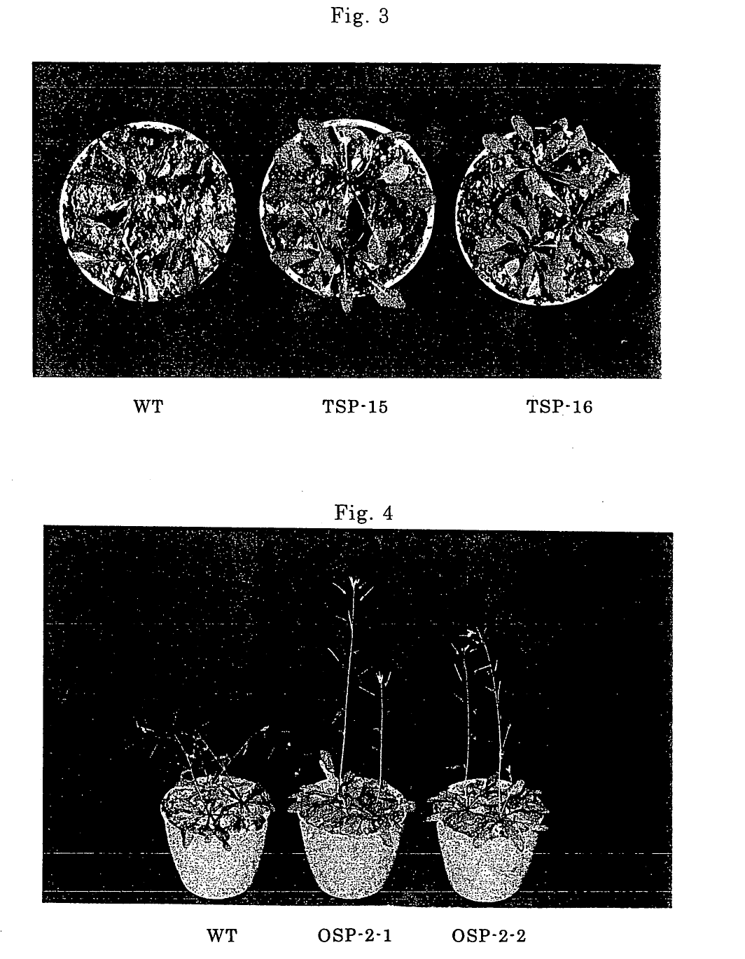

Withering from drought stress damage was noted in the wild type (WT) on day 13 after the start of drought treatment. 50% of the WT plants had died by Day 14 of drought treatment. Meanwhile, in the transformants, 20% of the plants died of drought treatment and higher survival rate than in the WT was shown. By Day 15, 100% of WT withered whereas 30 to 40% of the transformants survived. The results are given in FIG. 3. From the results in FIG. 3, it has been evidently confirmed that the wild type: WT (left) withered whereas the transformants: T3 homozygous cell lines (middle and right) survived.

Two transformants (T3 homozygous cell lines) selected from the transformant (OSP-2) were used. The seeds from the obtained two transformants and the wild type (WT: Columbia strain) were seeded in plastic pots containing the potting compost, Metromix (supplied from Hyponex Japan). Inoculation was followed by about 2 days of low temperature treatment at 4° C., and the pots were then placed in plastic vats to start cultivation at 23° C. under conditions involving long days (16 hour long days/8 hour nights). Rosette leaves had fully developed by about week 4 after inoculation. Individuals characterized by uniform growth at the time the rosette leaves had fully developed were selected, water was then fed into the vats to ensure uniform soil moisture, and water was filled to the middle of the plastic pots. After 5 days, a constant soil moisture was confirmed, and drought stress treatment was started (termination of water feed). The state of growth was monitored immediately after water termination.

Withering from drought stress damage was noted in the wild type (WT) on day 14 after the start of drought treatment. 50% of the WT plants had died by Day 15 of drought treatment. Meanwhile, in two transformants, 20% of the plants died of drought treatment, and the higher survival rate than in WT was shown. By Day 18 after the start of the treatment, 100% of WT withered whereas 50% of the transformants survived. The results are given in FIG. 4. From the results in FIG. 4, it has been evidently confirmed that the wild type: WT (left) withered whereas the transformants: T3 homozygous cell lines (middle and right) survived.

From the above results, it has been demonstrated that the drought stress defense effect can be imparted to the plant by introducing the spermidine synthase gene (FSPD1, OSPD2).

(3) Evaluation of Cold Stress Tolerance (Freeze Stress Tolerance)

The transformants (T3 homozygous cell lines) selected from the transformants (TSP-15, TSP-16) were used. The seeds from the obtained transformants and the wild type (WT: Columbia strain) were seeded in plastic pots containing the potting compost, Metromix (supplied from Hyponex Japan). For about 2 days after seeding, the cold temperature treatment at 4° C. was performed, subsequently the pots was placed in a plastic tray, and the cultivation was started at 23° C. under the long day condition (lighten for 16 hours and darken for 8 hours). Until about 4 weeks after seeding and until rosette leaves were completely developed, the plants were grown. At a time point when the rosette leaves were completely developed, individuals at the same growth stage were selected, and then transferred to a growth chamber at −5° C. to start the freeze stress treatment. The freeze stress treatment was performed in a dark phase for 40 hours. After the treatment, the plants were returned to the room at ambient temperature at 23° C. under the long day condition, and the state of growth was observed.

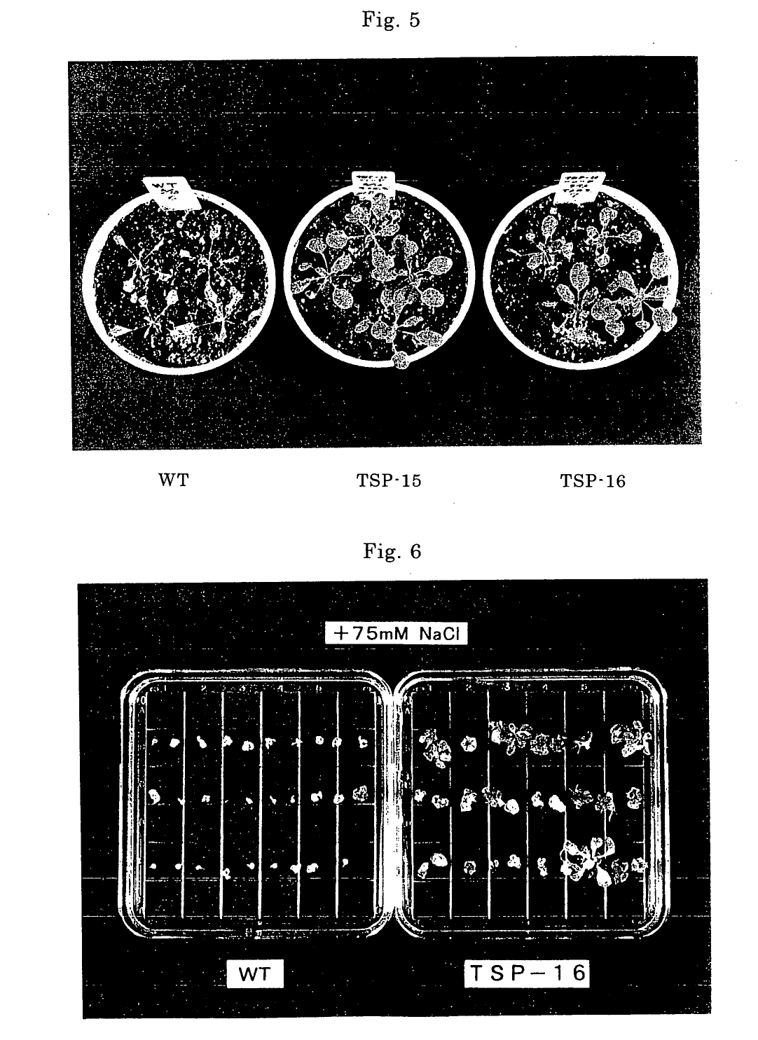

From immediately after returning to the ambient temperature, in the wild type (WT), a submerged state and wilting which were freeze stress disorders were observed. By Day 5 after returning to the ambient temperature, all plants withered in WT. Meanwhile, 30 to 40% of the plants survived in the transformants. The results were given in FIG. 5. From the results in FIG. 5, it has been evidently confirmed that the wild type: WT (left) withered whereas the transformants: T3 homozygous cell lines (middle and right) survived. Similar results were obtained in the cell line (OSP-2) in which the spermidine synthase gene (OSPD2) derived from the rice plant had been introduced.

From the above results, it has been demonstrated that the low temperature stress (freeze stress) defense effect can be imparted to the plant by introducing the spermidine synthase gene (SPDS) into the plant.

(4) Evaluation of Salt Stress Tolerance

The surfaces of seeds of the transformants (TSP-16) obtained in Example 2 and the wild type (WT: Columbia strain) were sterilized in the same manner as in section (6) of Example 2. Germination growth medium containing 75 mM NaCl (75 mM NaCl, 1×MS salt, 10 g/L sucrose, 0.1 g/L myo-inositol, 5% MES, 5 g/L Gellan gum, pH 5.7) was inoculated with the sterilized seeds one at a time. Inoculation was followed by about 2 days of low temperature treatment at 4° C., and then by the start of cultivation at 22° C. under conditions involving long days (16 hour long days/8 hour nights). In week 6 after inoculation, the extent of plant growth on the germination growth medium was observed. The results are given in FIG. 6.

From the results in FIG. 6, the remarkable growth inhibition was observed in WT which was control on the medium containing 75 mM NaCl, and the entire plant was whitened or yellowed to stop the growth and wither. Meanwhile, in the transformants in which the spermidine synthase gene had been introduced, the growth inhibition was given, but true leaves were developed and the growth continued as they were although it was late. Similar results were obtained in the cell line (OSP-2) in which the spermidine synthase gene (OSPD2) derived from the rice plant had been introduced.

From the above results, it has been demonstrated that the salt stress defense effect can be imparted to the plant by introducing the spermidine synthase gene (SPDS) into the plant.

(5) Evaluation of Herbicide Stress Tolerance

The surfaces of seeds (cell lines: pBI121 (35S-GUS), TSP-15, TSP-16, TSA-1, TAD-3) and the wild type (WT: Columbia strain) were sterilized in the same manner as in section (6) of Example 2. Germination growth medium containing 2 μM paraquat (PQ) (2 μM paraquat, 1×MS salt, 10 g/L sucrose, 0.1 g/L myo-inositol, 5% MES, 5 g/L Gellan gum, pH 5.7) was inoculated with the sterilized seeds one at a time. Inoculation was followed by about 2 days of low temperature treatment at 4° C., and then by the start of cultivation at 22° C. under conditions involving long days (16 hour long days/8 hour nights). The number of germinating individuals (germination rate) was observed on day 10 after inoculation, and the number of individuals surviving (survival rate) was observed on day 20. The results are given in Table 5.

TABLE 5

| Cell line | Germination rate | Survival rate |
|---|---|---|
| Wild type: WT | 3% | 3% |
| pBI121: 35S-GUS | 0% | 0% |
| TSP-15 | 50% | 25% |
| TSP-16 | 59% | 50% |
| TSA-1 | 48% | 32% |
| TAD-3 | 41% | 22% |

The results of Table 5 show that the wild type and vector control line (pBI121) had extremely low germination and survival rates as a result of toxicity caused by paraquat, whereas cell lines TSP-15, TSP-16, TSA-1, TAD-3 which contained polyamine synthase genes retained high germination and survival rates.

From the above results, it has been demonstrated that the herbicide stress defense effect can be imparted to the plant by introducing the polyamine synthases (SPDS, SAMDC, ADC) genes into the plant.

### EXAMPLE 5

### Production of Transgenic Sweet Potato

Sweet potato Kokei 14 (donated by Professor Takiko Shimada at Ishikawa Agricultural College, Agricultural Resource Institute, hereinafter referred to as “Kokei 14” or “wild type”) was grown and cultivated in a container under usual cultivation management to collect tens of cane tops (about 5 cm in length) containing shoot apex. The cane tops were immersed for two minutes in a 300-ml beaker to which 150 ml of ethanol had been added, followed by dipped for 2 minutes in a beaker to which 150 ml of disinfecting solution (5% sodium hypochlorite, 0.02% Triton X-100) had been added. Sterilized cane tops were washed with an aqueous sterilized solution placed in a sterilization beaker. After washing, about 0.5 mm of tissue containing meristematic tissue was aseptically removed under stereoscopic microscope. The tissue was then plated in embryogenic callus induction medium [4F1 plate:LS medium (1.9 g/l KNO₃, 1.65 g/l NH₄NO₃, 0.32 g/l MgSO₄.7H₂O, 0.44 g/l CaCl₂.2H₂O, 0.17 g/l KH₂PO₄, 22.3 mg/l MnSO₄.4H₂O, 8.6 mg/l ZnSO₄.7H₂O, 0.025 mg/l CuSO₄.5H₂O, 0.025 mg/l CoCl₂.6H₂O, 0.83 mg KI, 6.2 mg H₃BO₃, 27.8 mg FeSO₄.7H₂O, 37.3 mg/l Na₂-EDTA, 100 mg/l myo-inositol, 0.4 mg/l thiamine hydrochloride), 1 mg/L 4-fluorophenoxyacetic acid (4FA), 30 g/L sucrose, 3.2 g/l gellan gum, pH5.8] and then cultured in plant incubators (MLR-350HT, by Sanyo) under dark condition at 26° C. After one month culture, embryogenic calli capable of regeneration to plant body were selected from proliferated tissue. The selected embryogenic calli were continued to proliferate with transferred to a new 4F1 plate every month. Agrobacterium was infected as follows. Transformed Agrobacterium strains, EHA101/pBI35S-FSPD1+/−, EHA101/pBIC2-FSPD1+/− (CaMV35S promoter had been replaced with the peroxidase promoter derived from the horseradish), EHA101/pBI35S-FSAM24+/−, EHA101/pBIC2-FSAM24+/− (CaMV35S promoter had been replaced with the peroxidase promoter derived from the horseradish), EHA101/pBI35S-FADC76+/−, EHA101/pBIC2-FADC76+/− (CaMV35S promoter had been replaced with the peroxidase promoter derived from the horseradish) were cultured in LB medium containing 50 mg/L of kanamycin and 50 mg/L of hygromycin at 27° C. for two nights. Subsequently, about two rice grains of microbial cells were picked up, suspended in 50 mL of an infection medium (LS medium, 20 mg/L of acetosyringone, 1 mg/L of 4FA, 30 g/L of sucrose, pH 8), and shaken at 26° C. at 100 rpm under complete darkness for one hour. The suspension was transferred to 300-ml sterilized beaker in which stainless steel basket was placed. The embryogenic calli which were cultured two to three weeks were placed on the basket of the beaker for dipping for two minutes. The calli together with the basket were placed on doubled sterilized filter paper to remove the excess moisture. The calli were transferred on co-culture medium (4F1A20 plate: LS medium, 1 mg/l 4FA, 20 mg/l 3,5′-dimethoxy-4′-hydroxy-acetophenone, 30 g/l sucrose, 3.2 g/l gellan gum, pH5.8) and co-cultured for three days at 22° C. under dark condition. The embryogenic calli which were co-cultured for three days were transferred on the basket of the 300-ml beaker in which the sterilized stainless basket was placed. A 50 ml of disinfection solution containing carbenicillin in a final concentration of 500 mg/l in sterilized water was added to the beaker. The calli was fully washed for several minutes by anchoring the basket with tweezers. The embryogenic calli together with the basket were placed in the 300-ml beaker to which the disinfection solution was added for further washing. After repeating the same washing procedure, the excess moisture of the calli was removed on a sterilized filter paper to arrange and culture the calli in selection (4F1HmCar plate: LS medium, 1 mg/l 4FA, 25 mg/l hygromycin, 500 mg/l carbenicillin, 30 g/l sucrose, 3.2 g/l gellan gum, pH5.8) at 26° C. under dark condition. For selection of the transformed callus, the embryonic callus cultured for 2 weeks was cultured by transferring to a new 4F1HmCar plate every two weeks. Non-transformed calli turned brown, but part of transformants were embryogenic calli with pale yellow. After 60 days of culture on the selection medium, the transformed embryogenic calli were transferred to somatic cell embryogenic medium (A4G1HmCar plate: LS medium, 4 mg/l ABA, 1 mg/l GA3, 25 mg/l hygromycin, 500 mg/l carbenicillin, 30 g/l sucrose, 3.2 g/l gellan gum, pH5.8) to culture for two weeks at 26° C. under weak light and all long-day condition (30˜40 μmol/m²/s) and then transferred to plant body forming medium (A0.05HmCar plate: LS medium, 0.05 mg/l ABA 25 mg/l hygromycin, 500 mg/l carbenicillin, 30 g/l sucrose, 3.2 g/l gellan gum, pH5.8) to sulture in the same condition. The transformants were transferred to a new A0.05HmCar plate every two weeks. Since the transformed cells turned green to form somatic cell embryo derived from embryogenic calli, somatic cell embryo was transferred to plant grouth medium (0G plate: LS medium, 30 g/l sucrose, 3.2 g/l gellan gum, pH5.8) to form shoot. For the constructs in which FSAM24 and FADC76 had been controlled by the 35S promoter, the number of obtained transformants was obviously lower compared with the constructs controlled by the C2 promoter. Since the SAMDC gene and the ADC gene which act upstream of the polyamine metabolism largely affect the polyamine metabolism, when FSAM24 and FADC76 were controlled the constitutive promoter such as 35S promoter, it was likely that the excessive or rapid change of the polyamine amount adversely affected the growth. For the constructs in which FSAM24 and FADC76 had been controlled by the 35S promoter, many transformants as possible were assured, and among them, the transformants where the increase of the polyamine amount had been in the range of 1.1 to 4.0 times which had little or no effect on the growth and development were selected. The introduced gene was confirmed and the expression was analyzed for the obtained transformants. Specifically, to confirm the introduced gene, genomic DNA was prepared and then analyzed by PCR method and Southern hybridization. For the expression of the introduced gene, RNA was prepared, and then analyzed by Northern hybridization. As a result, the transformed sweet potatoes (transformants) in which the objective gene had been introduced and expressed could be obtained. Furthermore, the expression level and the translation level were examined in detail by Northern blotting and Western blotting, and cell lines in which the polyamine synthase gene had been introduced and the gene was stably expressed or translated were selected. Cell lines TSP-SS-1, TSP-SS-2, TSP-SS-3, TSP-SS-4, TSP-SS-5, TSP-SS-6. TSP-SS-7, TSP-CS-1, TSP-CS-3, TSP-CS-4 and TSP-CS-5 in which FSPD1 was introduced in a sense direction (+) were selected. TSP-SS-1, TSP-SS-2, TSP-SS-3, TSP-SS-4, TSP-SS-5, TSP-SS-6 and TSP-SS-7 were cell lines in which CaMV 35S promoter was introduced. TSP-CS-1, TSP-CS-3, TSP-CS-4 and TSP-CS-5 were cell lines in which a peroxidase promoter (C2 promoter) derived from horseradish was introduced. The cell lines, TSA-SS-1, TSA-SS-2, TSA-SS-5, TSA-SS-6, TSA-CS-1, TSA-CS-7 and TSA-CS-9 in which FSAM24 had been introduced in the sense direction were selected, TSA-SS-1, TSA-SS-2, TSA-SS-5 and TSA-SS-6 were the cell lines in which the CaMV35S promoter had been introduced as the promoter, and TSA-CS-1, TSA-CS-7 and TSA-CS-9 were the cell lines in which the peroxidase promoter (C2 promoter) derived from the horseradish had been introduced. The cell lines, TAD-SS-1, TAD-SS-2, TAD-SS-4, TAD-CS-1, TAD-CS-8 and TAD-CS-10 in which FADC76 had been introduced in the sense direction were selected, TAD-SS-1, TAD-SS-2 and TAD-SS-4 were the cell lines in which the CaMV35S promoter had been introduced as the promoter, and TAD-CS-1, TAD-CS-8 and TAD-CS-10 were the cell lines in which the peroxidase promoter (C2 promoter) derived from the horseradish had been introduced.

(2) Analysis of Polyamine

For the transformed sweet potatoes produced in (1), the cell lines controlled by the constitutive 35S promoter or the inducible C2 promoter were selected, and polyamine was analyzed under non-stress condition. Approximately 0.3 to 0.9 g of young leaves from the transgenic plants (TSP, TSA, TAD) and the wild type plants (WT), which were cultivated at the same time, were sampled, frozen and stored. Dilution internal standard solution (1,6-hexanediamine, internal standard content=7.5 or 12 nmol) and 5% perchloric acid aqueous solution (5 to 10 mL per 1.0 g specimen live body weight) were added to the sampled specimen, and was thoroughly ground down and extracted using an omnimixer at room temperature. The ground solution was centrifuged at 4° C., 35,000×g for 20 minutes, and the supernatant was collected and was taken as the free polyamine solution. Four hundred microliters of free polyamine solution, 200 μL of saturated sodium carbonate aqueous solution, and 200 μL of dansyl chloride/acetone solution (10 mg/mL) were added into a microtube with a screw cap, and lightly mixed. After firmly closing with a tube stopper and covering with aluminum foil, dansylation was conducted by heating for 1 hour in a 60° C. water bath. After allowing the tube to cool, 200 μL of proline aqueous solution (100 mg/mL) was added and mixed. The tube was covered with aluminum foil and heated again for 30 minutes in a water bath. After standing to cool, the acetone was removed by spraying nitrogen gas, and then 600 μL of toluene was added and vigorously mixed. After allowing the tube to stand quietly and separate into 2 phases, 300 μL of toluene in the upper layer was separated into a microtube. The toluene was completely removed by spraying nitrogen gas. 200 μL of methanol was added to the tube and the dansylated free polyamine was dissolved. The free polyamine content of putrescine, spermidine and spermine was assayed by the internal standard method using high performance liquid chromatography connected to a fluorescence detector (excitation wavelength: 365 nm·emission wavelength: 510 nm). A μBondapak C18 (manufactured by Waters, Co.: 027324, 3.9×300 mm, particle size 10 μm) was used for the HPLC column. The polyamine content in the specimens was calculated by deriving the peak areas of the polyamine and internal standard from the HPLC charts of the standard solution and specimens. A part of the results is shown in Table 6.

TABLE 6

| | Free polyamine content (nmolg⁻¹fw) | | | |
|---|---|---|---|---|
| | | | | Total |
| | | | | poly- |
| Cell line | Putrescine | Spermidine | Spermine | amines |
| Wild type: | 38.98 ± 12.53 | 78.65 ± 11.83 | 43.98 ± 5.78 | 161.61 |
| WT | | | | |
| TSP-SS-1 | 55.24 ± 5.71 | 148.91 ± 10.01 | 59.22 ± 3.57 | 263.37 |
| TSP-SS-2 | 49.98 ± 5.11 | 145.01 ± 12.17 | 65.19 ± 9.92 | 260.18 |
| TSP-SS-3 | 59.66 ± 3.98 | 140.94 ± 22.73 | 66.36 ± 6.48 | 266.96 |
| TSP-CS-1 | 57.66 ± 0.13 | 145.82 ± 3.62 | 74.24 ± 3.46 | 277.72 |
| TSP-CS-3 | 47.90 ± 4.86 | 122.78 ± 0.34 | 62.66 ± 5.71 | 233.34 |
| TSP-CS-4 | 52.73 ± 3.68 | 105.15 ± 1.30 | 54.79 ± 10.80 | 212.67 |
| TAD-SS-1 | 67.69 ± 1.12 | 115.27 ± 5.11 | 50.91 ± 4.18 | 233.87 |
| TAD-SS-2 | 66.14 ± 5.81 | 117.10 ± 10.02 | 52.04 ± 6.00 | 235.28 |
| TAD-SS-4 | 71.09 ± 3.68 | 107.98 ± 10.89 | 58.97 ± 9.81 | 238.04 |
| TSA-SS-1 | 63.37 ± 7.75 | 106.64 ± 16.47 | 61.94 ± 7.11 | 231.95 |
| TSA-SS-2 | 48.28 ± 6.68 | 134.06 ± 15.03 | 75.32 ± 13.13 | 257.66 |
| TSA-SS-5 | 57.78 ± 3.72 | 139.99 ± 12.93 | 59.54 ± 2.90 | 257.31 |

As is evident in Table 6, it has been demonstrated that the amounts of contained putrescine, spermidine and spermine were significantly increased and the total amounts of contained polyamine were also significantly increased in the cell lines (TSP-SS, TSA-SS and TSD-SS) in which the polyamine synthase gene (FSPD1, FSAM24 and FADC76) had been introduced in the sense direction under the control of the 35S promoter and the cell lines (TSP-CS) in which the gene had been introduced in the sense direction under the control of the C2 peroxidase promoter compared with the wild type (WT). Although, in TSA (FSAM24), the S-adenosylmethionine decarboxylase (SAMDC) gene largely affects the polyamine metabolism, the translation level was suppressed and no excessive increase of contained polyamine was observed because the SAMDC gene containing all 5′-non-translated region (uORF) had been introduced into the plant. In TAD (FADC76), the arginine decarboxylase (ADC) gene also largely affects the polyamine metabolism as with the SAMDC gene, but the translation level was suppressed and no excessive increase of contained polyamine was observed because the ADC gene containing all 5′-non-translated region (uORF) had been introduced into the plant.

### EXAMPLE 6

### Evaluation of Stress Tolerance of Transgenic Sweet Potato

(1) Evaluation of Salt Stress Tolerance

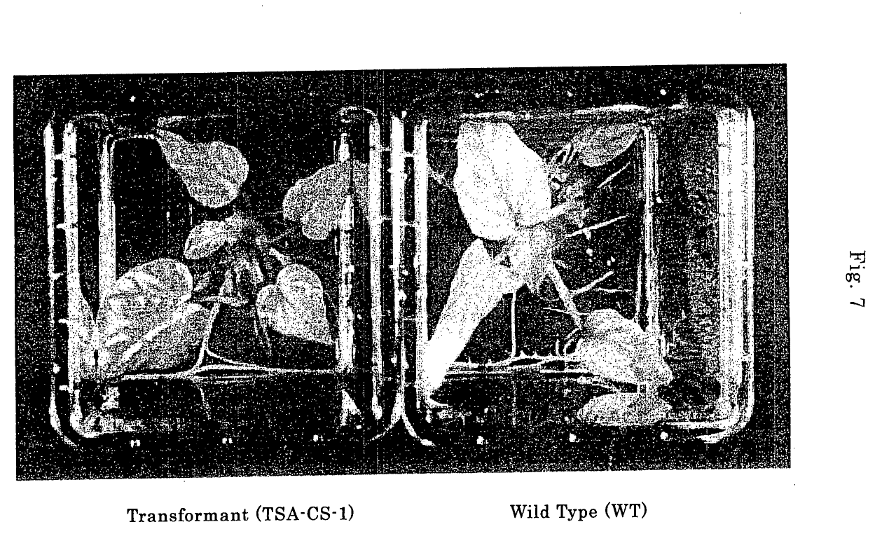

The cell lines (TSA-CS-1, TAD-CS-1) were selected in the transformed cell lines. Stem cuttings from two transformed cell lines and the wild type (WT) were planted to the growth medium (MS medium, 20 g/L of sucrose, 3.2 g/L of gellan gum, pH 5.8) in the presence or absence of high concentration of 150 mM NaCl, and transferred to the growth chamber (temperature at 25° C., lighten for 16 hours, 50 μmol m⁻² s⁻¹ PPFD) to start the salt stress study. One month after the start of the study, salt stress diseases were examined. As a result, in the leaves in the wild type (WT), a yellowing phenomenon which was one of the salt stress diseases was noticeably observed. Meanwhile, in the leaves in the two transformed cell lines (TSA-CS-1, TAD-CS-1), the yellowing phenomenon was not observed at all. A leaf sight of TSA-CS-1 was shown in FIG. 7. From the above results, it has been shown that growth disorder due to the salt stress is milder and the tolerance against the salt stress is remarkably excellent in the transformants in which the SAMDC gene or the ADC gene was excessively expressed under the control of the stress/disease-inducible promoter compared with wild type when encountered to the salt stress for a short period with higher salt concentration (150 mM) than the usual salt stress condition.

(2) Evaluation of Tolerance Against Moderate Environment (Low Temperature, Weak Light) Stress

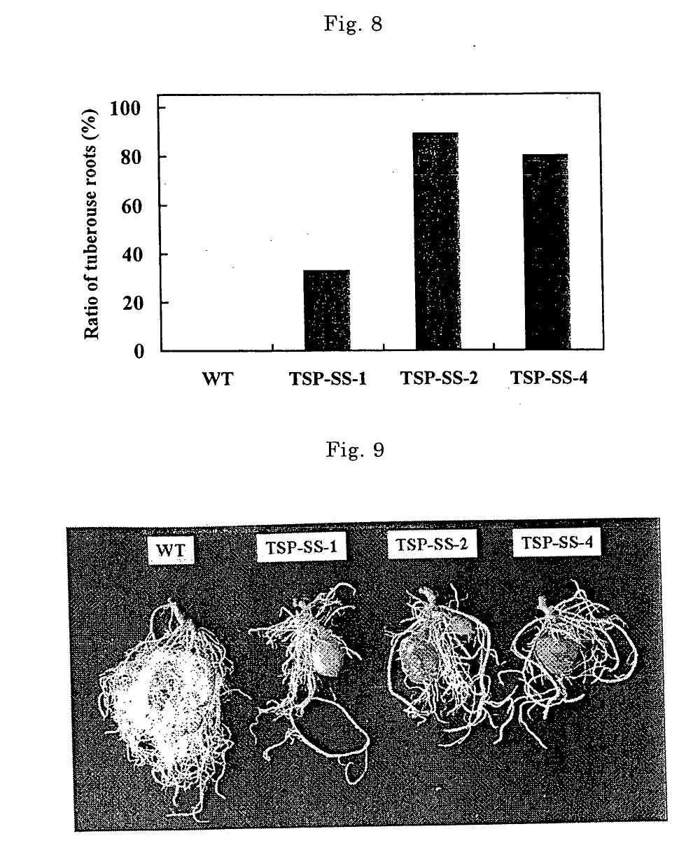

The cell lines (TSP-SS-1, TSP-SS-2, TSP-SS-4) were selected in the transformed cell lines. Stem cuttings (15 to 20 per line) from the three transformed lines and the wild type (WT) were planted in plastic pots filled with the commercially available potting compost (Sansan bed soil) to radicate. After radicating, the plants were cultivated in a closed system glass green house (temperature at 22 to 25° C., humidity at 55%, natural day length) for about one month until the fifth leaf was completely developed. Thereafter, the plants were transferred to the moderate environment of 21 to 22° C. and light intensity of 40 μmol m⁻² s⁻¹ PPFD (16 hours day length), and after 3 months, root tuber formation was surveyed. A formation rate of the root tubers and a root sight upon survey were shown in FIGS. 8 and 9, respectively (fine roots were removed to show the root tubers easily in the transformants). In the wild type, the root tuber was not formed at all. Meanwhile, the root tubers were formed in the transformants, and the formation rates of the root tubers in three transformants were 33, 80 and 89%, respectively. In a reproducibility study in which the plants were cultivated under the moderate environment of the same condition, the similar results were obtained. In the wild type, the root tuber was not formed at all, but the root tubers were formed in the transformants, and the formation rates of the root tubers were 50 to 83% depending on the cell lines.

(3) Evaluation of Tolerance Against Salt/Drought/Water Stresses

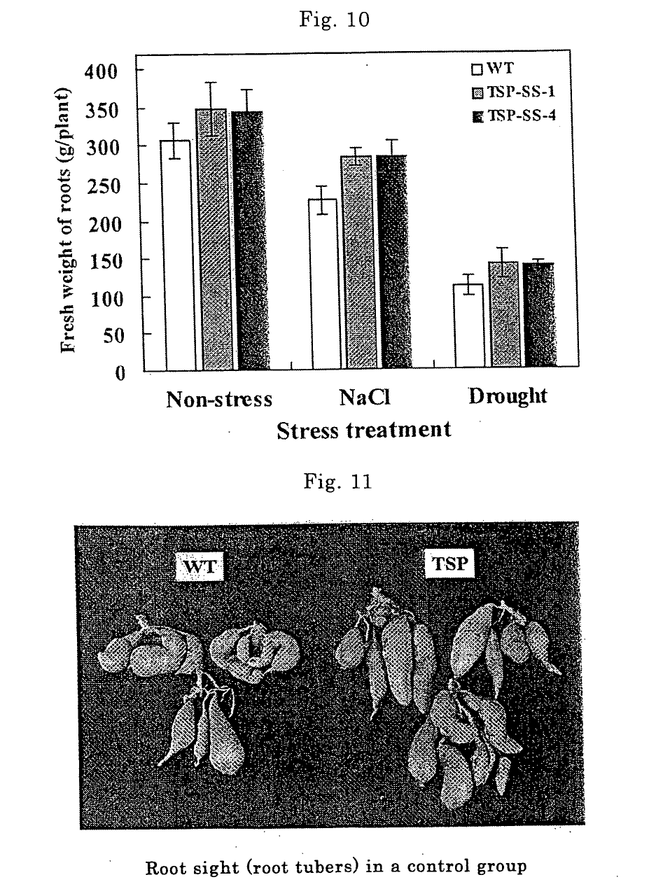

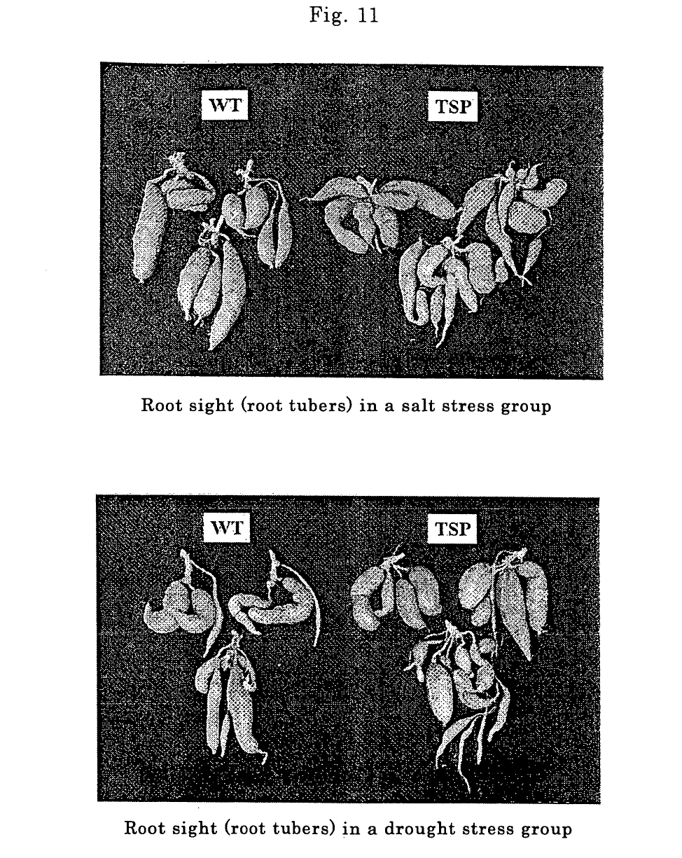

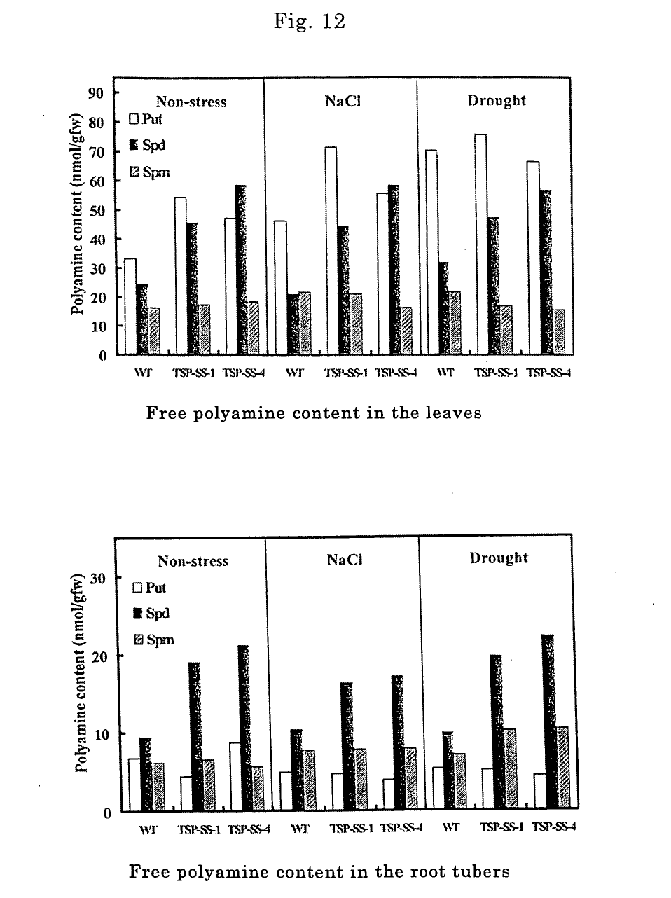

Two cell lines (TSP-SS-1, TSP-SS-4) were selected from the transformed cell lines. The two transformed cell lines and the wild type (WT: Kokei 14) were used to perform the cultivation experiment in the closed system glass green house. Stem cuttings with one sprout were prepared, and planted in the commercially available bed soil (Sansan bed soil) to radicate followed by being grown in the closed system glass green house (temperature at 23° C./21° C., humidity at 55%, natural day length) until the fifth leaf was completely developed. Seedlings at the same growth stage were selected in these seedlings after 3 weeks. Four stems were planted in each 6 per line of 30 L planters filled with 20 L potting compost (Sansan bed soil) (2 planters per treatment group), and cultivated in the closed system glass green house (set temperature at 23 to 24° C./21 to 22° C., humidity at 55%, natural day length). As fertilizers, 3.6 g of potassium sulfate and 13.4 g of Ecolong (14-12-14, 100 day type) per planter were given. Water in the soil was measured by placing a tensiometer (DM-8M supplied from Sansyo) in all planters. Groups were divided into a non-stress group (control group), a salt stress group and a drought stress group, and two planters were given to each group. In the salt stress group, 80 g of NaCl per 100 L of the potting compost was mixed with soil all layers when planted, and further 1.5 months after planting, 40 g of NaCl per 100 L of the potting compost was additionally given. The NaCl concentration was totally about 21 mmol/L per planter. The salt stress was started when fix planted. For affusion in the salt stress group and the control group, pF 2.3 of a soil water suction pressure was an affusion point, the amount of tap water (1.5 to 6 L/time per planter) where pF was lowered to a field capacity (pF 1.5) was given. The drought stress was given by limiting the affusion, and 0.75 to 3 L of water was given to the planter when pF became 2.9. The drought stress treatment was started one week after fix planting in consideration of taking roots after fix planting. About 4 months (harvesting stage) after fix plating, wet weight of the root tuber and the number of root tubers were surveyed. For polyamine analysis, the amounts of free polyamine contained in the leaves and the root tubers at the harvest were examined. The growth (wet weight) of the underground part (root tubers) by about 4 months after fix planting and a root sight were shown in FIGS. 10 and 11, respectively. The wet weight of the root tubers in the transformants was significantly heavier than in the wild type for the control group, and the root tuber weight per plant was about 40 g heavier in the transformants than in the wild type. The wet weight of the root tubers in the transformants was significantly heavier than in the wild type for the salt stress group, and the root tuber weight per plant was about 60 g heavier in the transformants than in the wild type. The wet weight of the root tubers in the transformants was significantly heavier than in the wild type for the drought group, and the root tuber weight per plant was about 30 g heavier in the transformants than in the wild type. Further, the numbers of the root tubers were shown in Table 5. In the transformants, the number of root tubers per plant was 2 more regardless of the presence and absence of the stress treatment. It was confirmed that the yield of the root tubers and the number of root tubers were obviously increased in the transformants compared with the wild type. Free polyamine in the leaf and the root tuber was analyzed 2 months after fix planting and at the harvest. The results at the harvest were shown in FIG. 12. Particularly, the amounts of spermidine (spd) contained in the leaf and the root tuber in the transformants were significantly increased about 2 times compared with the wild type regardless of the presence or absence of the stress treatment. Depending on the treatment group, the amounts of contained putrescine (Put) and spermine (Spm) were also increased. From the above results, under both non-stress condition and stress condition, it was shown that the formation of the root tubers (roots) was improved to increase the root tuber yield and the number of root tubers by increasing the polyamine level in the sweet potato. Next, the amount of contained starch which was a major component of the root tuber was examined. The root tuber with similar size was selected from each plant, and about 100 to 200 g of a root tuber piece was sampled from a vicinity of a center of the root tuber. The root tuber piece was cut finely, and 500 mL of distilled water was added to pulverize by a blender mixer for 1.5 minutes. A pulverization solution was filtrated with a sieve of 75 μm, and a filtrated solution was collected. The sieve was washed with 500 mL of distilled water and the filtrate was collected. The filtrated solution was left stand for several hours to precipitate starch, and a supernatant was discarded. Subsequently, 500 mL of distilled water was added, stirred to wash starch, and left stand to precipitate starch followed by discarding the supernatant. This manipulation was repeated three times or more to thoroughly wash starch. The precipitated starch after washing was dried in air at room temperature for 2 days or more. The dried starch was collected and weighed to calculate the amount of contained starch per wet weight and a starch yield (g/plant). As a result, in all treatment groups, the amount of contained starch per root tuber wet weight was higher in the transformants than in the wild type, and particularly, in the drought stress group, the amount of contained starch in the wild type was 12% whereas that in the both transformants (TSP-SS-1, TSP-SS-4) was 17% which was high. The starch yields were 39.6 g/plant in the wild type, 48.3 g/plant in TSP-SS-1 and 49.8 g/plant in TSP-SS-4 in the salt stress group, as well as 13.8 g/plant in the wild type, 21.1 g/plant in TSP-SS-1 and 21.8 g/plant in TSP-SS-4 in the drought stress group. In both treatment groups, the starch yield was higher in the transformants than in the wild type. From the above, it has been demonstrated that the amount of contained-starch which is the major component of the root tuber and the starch yield are increased by increasing the polyamine level in the plant.

According to the present invention, by being capable of imparting the stress defense effects of the plant, it is possible to avoid the diseases due to various stresses which the plant encounters in its growth and development process and reduce the growth inhibition, it is also possible to anticipate the stabilization of cultivation, the enhancement of productivity, the enlargement of cultivation regions and the enlargement of cultivation periods, and it is anticipated to largely contribute to the industry. It becomes possible to cultivate the plants in barren lands and salt accumulated soils, and it can be anticipated to contribute to the global warming and the food problem.

1. A method of inducing an expression of at least two stress defense genes in a plant, wherein an expression amount of at least one stress defense gene is increased compared with a non-transformant by transforming the plant with an exogenous spermidine synthase (SPDS) gene, an exogenous S-adenosylmethionine decarboxylase (SAMDC) gene, an exogenous arginine decarboxylase (ADC) gene, an ornithine decarboxylase (ODC) gene and/or a spermine synthase (SPMS) gene under the control of a promoter capable of functioning in the plant.

2. The method according to claim 1 further including a step of selecting a transformed plant in which expression levels of at least two stress defense genes have been increased compared with the non-transformant.

3. The method according to claim 1 wherein the stress defense gene is selected from the group consisting of 49 genes with specific Accession Number selected from the group consisting of the followings and genes having 60% or more homology to these genes.

| Gene | | Accession |
|---|---|---|
| Number | Stress defense gene | Number |
| 1 | transcription factor/CBF1, DREB1B | AT4G25490 |
| 2 | cold regulated protein/LEA protein | AT2G03740 |
| 3 | cold regulated protein/cor15 | AT2G42530 |
| 4 | pathogen related PR-1 protein/PR-1 | AT2G14610 |
| 5 | early response dehydration protein/ERD15 | D30719 |
| 6 | salt stress induced tonoplast intrinsic | AF004393 |
| | protein/aquaporin | |
| 7 | water channel protein/aquaporin | AAC79629 |
| 8 | dehydration induced protein/RD22, rd22 | AT5G25610 |
| 9 | stress responsive protein | CAB52439 |
| 10 | drought induced protein | AT1G72290 |
| 11 | low temperature and salt responsive protein | CAB79783 |
| 12 | stress responsive protein | CAB52439 |
| 13 | zinc finger protein | AT5G43170 |
| 14 | disease resistance protein | BAB08633 |
| 15 | disease resistance protein | AT3G05660 |
| 16 | disease resistance protein | BAB09430 |
| 17 | disease resistance protein | AT5G18350 |
| 18 | disease resistance protein | AT4G11210 |
| 19 | Peroxidase | AT4G33420 |
| 20 | senescence associated protein sen1 | AT4G35770 |
| 21 | senescence associated protein | BAB33421 |
| 22 | nematode resistance protein/Hs1pro-1 | NP181529 |
| 23 | WRKY transcription factor | AT4G23810 |
| 24 | RMA1 RING zinc finger protein | BAA28598 |
| 25 | Transcriptional activator CBF1/CBF1 | AT1G12630 |
| 26 | zinc finger protein | AT3G07650 |
| 27 | transcription factor/DREB1A | AT1G63030 |
| 28 | transcription factor/DREB1A | AT1G63040 |
| 29 | stress induced protein sti1 | T48150 |
| 30 | early response dehydration protein/ERD15 | T02438 |
| 31 | B-box zinc finger protein | AT1G68520 |
| 32 | transcription factor/DREB2B | AT3G11020 |
| 33 | heat shock protein DnaJ homolog | AT4G36040 |
| 34 | pathogenesis related protein | T04989 |
| 35 | Myb protein | AT4G372760 |
| 36 | jasmonic acid regulatory protein | AAF35416 |
| 37 | early response dehydration protein/ERD15 | D30719.1 |
| 38 | Low temperature induced protein 78/LTI78, | AT5G52310 |
| | rd29A, COR78 | |
| 39 | salt tolerance zinc finger protein | CAA64820 |
| 40 | CCCH type zinc finger protein | AT2G25900 |
| 41 | Cytochrome P450 | AT5G45340 |
| 42 | transcription activator CBF1/CBF1 | AT1G12610 |
| 43 | DREB like AP2 domain transcription | AT2G38340 |
| | factor/DREB2E | |
| 44 | Peroxidase | AT5G64120 |
| 45 | AP2 domain protein | AT1G78080 |
| 46 | senescence associated protein sen1 | AT4G35770 |
| 47 | stress responsive protein | CAB52439 |
| 48 | zinc finger protein | AT5G43170 |
| 49 | disease resistance protein | BAB08633 |

4. The method according to claim 1 wherein the stress defense gene is selected from the group consisting of the following:
I. CBF1, DREB1B
II. CBF3, DREB1A
III. DREB2B
IV. LTI78, COR78, rd29A
V. RD22, rd22
VI. Cor15
VII. ERD15
VIII. LEA protein
IX. PR-1
X. Peroxidase
XI. Hslpro-1

5. The method according to claim 1 wherein the expression amount of the stress defense gene is augmented 1.3 to 10 times compared with a plant before being transformed.

6. The method according to claim 1 wherein the expression amount of the stress defense gene is augmented 1.4 to 8 times compared with the non-transformant.

7. The method according to claim 1 wherein the expression amount of the stress defense gene is augmented 1.5 to 6 times compared with the non-transformant.

8. The method according to claim 1 wherein a gene introduced into the plant is the exogenous spermidine synthase (SPDS) gene derived from a plant.

9. The method according to claim 1 wherein the exogenous spermidine synthase (SPDS) gene is a spermidine synthase gene having a base sequence of the following (a) or (b) or (c):
(a) a base sequence represented by base numbers 77 to 1060 in a base sequence represented by SEQ ID NO:1 (SPDS, 1328);
(b) a base sequence which hybridizes with the above base sequence (a) or a complementary chain thereto under a stringent condition and encodes a protein having a spermidine synthase activity; and
(c) a base sequence which is composed of a sequence having one or more base deletions, substitutions, insertions or additions in the base sequence (a) or (b) and encodes the protein having the spermidine synthase activity.

10. The method according to claim 1 wherein the exogenous spermidine synthase (SPDS) gene is a spermidine synthase gene having a base sequence of the following (a) or (b) or (c):
(a) a base sequence represented by base numbers 118 to 1281 in a base sequence represented by SEQ ID NO:3 (SPDS, 1560);
(b) a base sequence which hybridizes with the above base sequence (a) or a complementary chain thereto under a stringent condition and encodes a protein having a spermidine synthase activity; and
(c) a base sequence which is composed of a sequence having one or more base deletions, substitutions, insertions or additions in the base sequence (a) or (b) and encodes the protein having the spermidine synthase activity.

11. The method according to claim 1 wherein the exogenous S-adenosylmethionine decarboxylase (SAMDC) gene is an S-adenosylmethionine decarboxylase gene having a base sequence of the following (a) or (b) or (c):
(a) a base sequence represented by base numbers 456 to 1547 in a base sequence represented by SEQ ID NO:5 (SAMDC, 1814);
(b) a base sequence which hybridizes with the above base sequence (a) or a complementary chain thereto under a stringent condition and encodes a protein having a S-adenosylmethionine decarboxylase activity; and
(c) a base sequence which is composed of a sequence having one or more base deletions, substitutions, insertions or additions in the base sequence (a) or (b) and encodes the protein having an S-adenosylmethionine decarboxylase activity.

12. The method according to claim 1 wherein the exogenous arginine decarboxylase (ADC) gene is an arginine decarboxylase gene having a base sequence of the following (a) or (b) or (c):
(a) a base sequence represented by base numbers 541 to 2661 in a base sequence represented by SEQ ID NO:7 (ADC, 3037);
(b) a base sequence which hybridizes with the above base sequence (a) or a complementary chain thereto under a stringent condition and encodes a protein having a arginine decarboxylase activity; and
(c) a base sequence which is composed of a sequence having one or more base deletions, substitutions, insertions or additions in the base sequence (a) or (b) and encodes the protein having an arginine decarboxylase activity.

13. The method according to claim 1 wherein the exogenous spermine synthase (SPMS) gene is a spermine synthase gene having a base sequence of the following (a) or (b) or (c):
(a) a base sequence represented by base numbers 1 to 1020 in a base sequence represented by SEQ ID NO:9 (SPMS, 1020);
(b) a base sequence which hybridizes with the above base sequence (a) or a complementary chain thereto under a stringent condition and encodes a protein having a spermine synthase activity; and
(c) a base sequence which is composed of a sequence having one or more base deletions, substitutions, insertions or additions in the base sequence (a) or (b) and encodes the protein having a spermine synthase activity.

14. The method according to claim 1 wherein an introduced polyamine synthase gene is a gene encoding an arginine decarboxylase (ADC) and/or a gene encoding an S-adenosylmethionine decarboxylase (SAMDC) comprising uORF upstream of the gene.

15. The method according to claim 1 wherein one or more stress defense effects selected from the group consisting of the following (i) to (xiii) can be imparted:
(i) low temperature stress
(ii) high temperature stress
(iii) salt stress
(iv) osmotic stress
(v) oxidative stress
(vi) herbicide stress
(vii) freeze stress
(viii) drought stress
(ix) pathogen infection stress
(x) pest stress
(xi) disease stress
(xii) aging stress and
(xiii) heavy metal stress.

16. A method of imparting stress defense effects to a plant wherein expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an ornithine decarboxylase (ODC) gene and/or a spermine synthase (SPMS) gene under the control of a promoter capable of functioning in the plant.

17. A method of imparting stress defense effects to a plant wherein expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an ornithine decarboxylase (ODC) gene and/or a spermine synthase (SPMS) gene under the control of a promoter capable of functioning in the plant, and a transformed plant in which expression levels of the stress defense genes have been increased compared with a non-transformed plant (wild type) is selected

18. A method of enhancing productivity of a plant wherein expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an ornithine decarboxylase (ODC) gene and/or a spermine synthase (SPMS) gene under the control of a promoter capable of functioning in the plant.

19. A method of enhancing stress tolerance in a plant wherein expression amounts of at least two stress defense genes are increased compared with a non-transformant by transforming the plant with an exogenous spermidine synthase (SPDS) gene, an exogenous S-adenosylmethionine decarboxylase (SAMDC) gene, an exogenous arginine (ADC) decarboxylase gene, an ornithine decarboxylase (ODC) gene and/or a spermine synthase (SPMS) gene under the control of a promoter capable of functioning in the plant.
